(19) United States
(12) Patent Application Publication
(10) Pub. No.: US 2015/0066042 A1
(43) Pub. Date: Mar. 5, 2015

(54) MINIMALLY INVASIVE DEVICES AND METHODS FOR DELIVERING FIXATION DEVICES AND IMPLANTS INTO A SPINE

(71) Applicant: Spinal USA, Inc., Parsippany, NJ (US)

(72) Inventors: John Franklin Cummins, North Kosciusko, MS (US); William Francis Xavier Fahey, III, Ridgewood, NJ (US)

(21) Appl. No.: 14/374,882

(22) PCT Filed: Jan. 24, 2013
(86) PCT No.: PCT/US2013/022896

§ 371 Date: Jul. 25, 2014

Related U.S. Application Data
(60) Provisional application No. 61/591,248, filed on Jan. 26, 2012.
(60) Provisional application No. 61/653,853, filed on May 31, 2012.
(60) Provisional application No. 61/590,789, filed on Jan. 25, 2012.

(51) Int. Cl. A61B 17/70

(52) U.S. Cl. CPC A61B 17/7074; A61B 17/7085; A61B 17/7082; A61B 17/7083
USPC 606/104; 606/86 A

(57) ABSTRACT

Devices and methods are provided for assisting with spinal stabilization. One or more spinal stabilization systems can be coupled to one or more tower access devices for delivery to target locations in a patient. The spinal stabilization systems can include a bone screw, a housing for the bone screw and a spinal rod. A screw delivery device can be inserted through the tower access device and used to anchor the bone screws to the spine. A rod delivery device can be used to insert the rod along the tower access devices to the housings. A rod reducer can thread onto the tower access device and urge the rod into position. A hinge can couple two tower access devices at a desired lateral spacing while allowing some pivotal movement of the access devices. Systems, kits, and methods combining and utilizing the aforementioned devices are also provided.

### CROSS-REFERENCE TO RELATED APPLICATIONS

This application claims priority benefit of U.S. Provisional Application Nos. 61/590,789, 61/591,248, and 61/653,853, entitled “MINIMALLY INVASIVE DEVICES AND METHODS FOR DELIVERING FIXATION DEVICES AND IMPLANTS INTO A SPINE,” filed Jan. 25, 2012, Jan. 26, 2012, and May 31, 2012, respectively, the entireties of all of which are hereby incorporated by reference.

### BACKGROUND

1. Field

The present application relates to devices and methods for providing spinal stabilization. In particular, the present application relates to minimally invasive devices and methods for delivering fixation devices and implants into a spine.

2. Description of the Related Art

Spinal bone degeneration can occur due to trauma, disease or aging. Such degeneration can cause abnormal positioning and motion of the vertebrae, which can subject nerves that pass between vertebral bodies to pressure, thereby causing pain and possible nerve damage to a patient. In order to alleviate the pain caused by bone degeneration, it is often helpful to maintain the natural spacing between vertebrae to reduce the pressure applied to nerves that pass between vertebral bodies.

To maintain the natural spacing between vertebrae, spinal stabilization devices are often provided to promote spinal stability. These spinal stabilization devices can include fixation devices, such as spinal screws, which are implanted into vertebral bone. The fixation devices work in conjunction with other implanted members, such as rod members, to form stabilization systems.

Conventional stabilization systems often require open surgeries and other invasive procedures in order to deliver the implants into the body. These invasive procedures often cause a great deal of pain and trauma to the patient, and require a substantial recovery time. Thus, there exists a need for minimally invasive devices and methods that can assist in providing spinal stabilization.

### SUMMARY

In some embodiments, a minimally invasive spine surgery access device includes an elongate body having a proximal end, a distal end, and an inner lumen extending there through. The access device includes a threaded portion proximate the proximal end, two grasping elements at the distal end, and a longitudinal slot along at least one side of the elongate body. In some embodiments, the access device includes a spring latch attached to the elongate body. The spring latch has a tab that extends into the threaded portion and is moveable between an elevated position and a depressed position. The access device further includes a hollow and internally threaded lock nut configured to thread onto the threaded portion. An inner perimeter of a distal end of the lock nut includes a series of ramps configured to engage the spring latch tab to inhibit counter rotation of the lock nut. Advancing the lock nut distally on the threaded portion causes compression of the grasping elements, and movement of the spring latch tab to the depressed position disengages the tab from the lock nut ramps to permit counter rotation of the lock nut.

In some embodiments, a minimally invasive spine surgery access device includes an elongate inner sleeve and elongate outer sleeve. The inner sleeve includes an externally threaded portion at a proximal end and a pair of distal slots located on opposite sides of the inner sleeve extending proximally from a distal end at least partially along a length of the inner sleeve. The outer sleeve slidably receives the inner sleeve, and the outer sleeve includes a pair of distal slots located on opposite sides of the outer sleeve and extending proximally from a distal end at least partially along a length of the outer sleeve. When assembled, the distal slots of the outer sleeve are substantially aligned with the distal slots of the inner sleeve. The outer sleeve also includes a proximal slot extending from a proximal end of the outer sleeve at least partially along a length of the outer sleeve. The distal end of the inner sleeve includes two grasping elements that extend distally past the distal end of the outer sleeve and include protrusions or pins configured to grasp a spinal implant. Proximal movement of the inner sleeve relative to the outer sleeve causes compression of the grasping elements, and distal movement of the inner sleeve relative to the outer sleeve causes expansion of the grasping elements. The access device includes a spring latch attached to the inner sleeve and positioned within the proximal slot of the outer sleeve. A distal portion of the spring latch is attached to the inner sleeve, and a proximal end of the spring latch includes a tab and a button distal to the tab. The proximal end of the spring latch is moveable between and an elevated position and a depressed position. The access device further includes a hollow and internally threaded lock nut configured to thread onto the threaded portion of the inner sleeve. Threading of the lock nut onto the threaded portion causes the lock nut to engage the proximal end of the outer sleeve and move the inner sleeve proximally relative to the outer sleeve to cause compression of the grasping elements. An inner perimeter of a distal end of the lock nut includes a series of ramps having shoulders of about 90°, and the ramp shoulders are configured to engage the spring latch tab to inhibit counter rotation of the lock nut. The button on the spring latch is moveable to its depressed position to disengage the spring latch tab from the lock nut ramps to permit counter rotation of the lock nut and to permit distal movement of the inner sleeve relative to the outer sleeve. The relative positions of the inner and outer sleeves are in a locked configuration when the lock nut is engaged with the spring latch tab and the grasping elements are in a compressed configuration, and the relative positions of the inner and outer sleeves are in an unlocked configuration when the ramps of the lock nut are not engaged with the spring latch tab and the inner and outer sleeves are moveable relative to one another to permit compression and expansion of the grasping elements.

In some embodiments, a screw delivery device includes a shaft, a grip, a tip configured to engage a portion of a bone screw, and a saddle configured to engage another portion of a screw assembly to help distribute load during tightening of the bone screw.

In some embodiments, a screw delivery device includes an inner elongate shaft and an outer elongate shaft. The inner elongate shaft has a proximal end and a distal end, the distal end of the inner shaft including a hexalobe tip configured to engage a first portion of a spinal screw and a saddle proximal to the hexalobe tip configured to engage a second portion of the spinal screw. The outer elongate shaft is provided over the inner elongate shaft and is axially and rotationally moveable relative the inner shaft. The outer shaft has a proximal end and a distal end, the proximal end of the outer shaft including a grip and the distal end of the outer shaft including an externally threaded portion configured to engage an internally threaded third portion of the spinal screw.

In some embodiments, a system includes a screw delivery device and a screw assembly. The screw assembly includes, a housing having a lower opening and internal threads configured to receive an externally threaded set screw, a threaded shaft having an enlarged head, the threaded shaft extending through the lower opening and the enlarged head sitting adjacent the lower opening, and a rod receiving surface within the housing having a concave shape configured to receive a rod. The threaded shaft is rotatable relative to the housing prior to insertion of the set screw. The hexalobe tip of the screw delivery device is configured to engage a corresponding opening in the enlarged head of the screw assembly, the saddle is configured to engage the rod receiving surface, and the externally threaded portion at the distal end of the outer shaft is configured to engage the internal threads of the housing.

In some embodiments, a method of delivering a screw assembly to a spinal location includes positioning a screw assembly at a desired spinal location. The screw assembly includes a housing having a lower opening and internal threads configured to receive an externally threaded set screw, a threaded shaft having an enlarged head, the threaded shaft extending through the lower opening and the enlarged head sitting adjacent the lower opening, and a rod receiving surface within the housing having a concave shape configured to receive a rod. The threaded shaft is rotatable relative to the housing prior to insertion of the set screw. The method includes engaging a screw delivery device with the screw assembly at the spinal location. The screw delivery device includes an inner elongate shaft having a proximal end and a distal end, the distal end of the inner shaft comprising a hexalobe tip configured to engage a corresponding opening in the enlarged head of the screw assembly and a saddle proximal to the hexalobe tip configured to engage the rod receiving surface and an outer elongate shaft provided over the inner elongate shaft, the outer shaft being axially and rotationally moveable relative the inner shaft, the outer shaft having a proximal end and a distal end, the proximal end of the outer shaft comprising a grip and the distal end of the outer shaft comprising an externally threaded portion configured to engaged the internal threads of the housing. The method further includes rotating the outer shaft relative to the inner shaft while the hexalobe tip engages the enlarged head of the screw assembly to cause the externally threaded portion at the distal end of the outer shaft to engage the internal threads of the housing and rotating the screw delivery device while the inner and outer shafts remain relatively locked in position to drive the threaded shaft into the spinal location, said rotation rotating the hexalobe tip against the opening in the enlarged head of the screw assembly with the saddle distributing load to the rod receiving surface during said rotation.

In some embodiments, a rod insertion device includes an elongate, generally cylindrical shaft and a gripping end at a distal end of the shaft. The gripping end includes two or more gripping elements configured to grip a rod member. The device further includes a handle at a proximal end of the shaft and an actuator member configured to cause relative movement between the shaft and gripping end. Distal movement of the shaft or proximal movement of the gripping end causes compression of the gripping elements. In some embodiments, the gripping elements include serrations configured to mate with serrations on the rod member.

In some embodiments, a rod for use for spinal fixation includes an elongate cylindrical member having a first end and a second end, wherein one of the ends comprises a plurality of serrations.

In some embodiments, a method of delivering a rod to a spinal location includes providing a rod having a serrated portion and providing a rod insertion device that includes an elongate, generally cylindrical shaft having a gripping end at a distal end of the shaft and a handle at a proximal end of the shaft. In some embodiments, the gripping end includes two or more gripping elements configured to grip the rod member and the gripping elements include serrations. The method further includes grasping the rod with the rod insertion device by engaging the serrations of the gripping elements and serrated portion of the rod and delivering the rod to the spinal location with the rod insertion device.

In some embodiments, a rod reducer includes a cannula having a proximal end and a distal end and a slot at the distal end configured to interact with a rod member. An internally threaded portion is coupled to the proximal end of the cannula so that the cannula can remain substantially rotationally fixed while the threaded portion rotates.

In some embodiments, a method of positioning a rod member within a screw assembly at a spinal location includes providing a rod reducer that includes a cannula having a proximal end and a distal end, an internally threaded portion coupled to the proximal end, and a slot at the distal end configured to interact with a rod member. The method includes advancing the rod reducer over a tower access device having a distal end positioned at the spinal location and a proximal end positioned outside of the patient, the proximal end of the tower access device having an externally threaded portion, engaging a rod member positioned within the screw assembly with the slot at the distal end of the rod reducer, and distally threading the internally threaded portion of the rod reducer to the externally threaded portion of the tower access device by rotating the internally threaded portion, wherein said distal threading urges the rod into engagement with the screw assembly, wherein said cannula and the slot remain rotationally fixed relative to the rod member as the internally threaded portion is rotated.

In some embodiments, a hinge for connecting two or more spinal access tower devices includes at least two clamps configured to engage two tower access devices. Each clamp includes a pivot having a spherical outer surface and a cylindrical bore therethrough, the cylindrical bore configured to receive a tower device. Each clamp also includes a clamshell clamp having a cylindrical outer surface and spherical inner surface circumferentially surrounding the pivot and a clamp tightener configured to tighten the clamshell clamp around the pivot. In use, the spherical outer surfaces of the pivots allow for pivotal movement relative to the clamshell clamps. The hinge further includes a ratchet bar coupling the two clamps and a tightening knob configured to lock the ratchet bar to fix the lateral spacing between the clamps.

In some embodiments, a method for connecting two or more spinal access tower devices includes positioning at least two clamps around at least two adjacent tower devices, respectively. In some embodiments, each clamp includes a pivot having a spherical outer surface and a cylindrical bore therethrough, the cylindrical bore configured to receive a tower device and a clamshell clamp circumferentially surrounding the pivot, the clamshell clamp having a cylindrical outer surface and a spherical inner surface. The method further includes tightening the clamshell clamps around each of the pivots to secure the at least two adjacent tower devices, wherein the spherical outer surfaces of the pivots allow for pivotal movement of the tower devices relative to the clamshell clamps, and adjusting a lateral spacing between the clamps by moving one or more of the clamps along a ratchet bar coupling the two clamps.

In some embodiments, a system for fixation of the spine includes: one or more tower access devices having a proximal end configured to extend outside of a patient and a distal end configured to engage a spinal screw; a screw delivery device configured to be delivered through the one or more tower access devices and configured to tighten the screw, wherein the screw delivery device comprises a saddle configured to engage a portion of the screw to distribute load during tightening; a rod inserter configured to deliver a rod through the one or more tower access devices to the screw; and a threaded reducer configured to engage a tower access device, the threaded reducer having a slot configured to engage the rod while the tower access device engages the screw. In further embodiments, one or more dilation tubes configured to provide access to a portion of a patient's spine may be provided. In further embodiments, an awl and/or a tap configured to prepare the portion of the patient's spine to receive a screw may be provided. Further embodiments comprise a compressor, a distractor, and/or other devices described herein. Such devices can be provided in a single kit or a single tray.

### BRIEF DESCRIPTION OF THE DRAWINGS

FIG. 1 shows an exploded view of an embodiment of a spinal stabilization system;

FIG. 2 shows a partially assembled view of the spinal stabilization system of FIG. 1;

FIG. 3 shows a perspective view of an embodiment of a minimally invasive tower access device;

FIG. 4A shows a side view of the minimally invasive tower access device of FIG. 3;

FIG. 4B shows an exploded view of the minimally invasive tower access device of FIGS. 3 and 4A;

FIG. 5 shows an embodiment of a spring latch of the minimally invasive tower access device of FIG. 3;

FIG. 6 shows an embodiment of a lock nut of the minimally invasive tower access device of FIG. 3;

FIG. 7 shows the spinal stabilization system of FIG. 1 coupled to the minimally invasive tower access device of FIG. 3;

FIGS. 8A-8D show dilators having increasing dimensions according to embodiments of the present disclosure;

FIG. 9 shows an awl according to embodiments of the present disclosure;

FIG. 10 shows a tap according to embodiments of the present disclosure;

FIGS. 11A-11B show perspective views of an embodiment of a screw delivery device;

FIG. 11C shows an exploded view of the screw delivery device of FIG. 11A;

FIG. 11D shows a detail view of a distal end of the screw delivery device of FIG. 11A;

FIG. 12A shows a side view of an embodiment of a rod insertion device;

FIG. 12B shows a perspective view of an outer shaft and handle of the rod insertion device of FIG. 12A;

FIG. 12C shows a perspective view of an inner shaft of the rod insertion device of FIG. 12A;

FIG. 12D shows a detailed view of a distal end of the inner shaft of FIG. 12C;

FIGS. 12E-12F show views of a serrated rod;

FIG. 12G shows a perspective view of the rod insertion device of FIG. 12A engaging the rod of FIG. 12E;

FIG. 12H shows a detail view of a distal end of the rod insertion device of FIG. 12A engaging the rod;

FIGS. 13A-13B show front perspective views of a threaded rod reducer according to embodiments of the present disclosure;

FIG. 13C shows a front perspective view of a top portion of the threaded rod reducer of FIGS. 13A-13B;

FIG. 14 shows a perspective view of an embodiment of a set screw inserter;

FIG. 15 shows a perspective view of an embodiment of a hinge;

FIG. 16 shows a front view of an example caliper;

FIG. 17 shows a perspective view of an example compressor;

FIG. 18 shows a perspective view of an example distracter;

FIG. 19 shows a perspective view of an example final tightener;

FIG. 20 shows a dilation tube inserted over a K-wire;

FIG. 21 shows an awl inserted through a dilation tube and over a K-wire;

FIG. 22 shows a tap inserted through a dilation tube and over a K-wire;

FIG. 23 shows the minimally invasive tower access device of FIG. 3 coupled to the spinal stabilization system of FIG. 1 and inserted through a dilation tube over a K-wire;

FIG. 24 shows the screw delivery device of FIGS. 11A and 11B inserted through the assembly of FIG. 23;

FIG. 25 shows the system of FIG. 25 with the dilation tube removed;

FIG. 26 shows the rod insertion device of FIG. 12A coupled to a rod with the tower access device of FIG. 3 coupled to the spinal stabilization system of FIG. 1;

FIGS. 27-28 show rod reduction;

FIG. 29 shows set screw insertion;

FIG. 30 shows a pair of spinal stabilization system and tower access device assemblies coupled by a rod and hinge;

FIG. 31 shows compression;

FIG. 32 shows distraction;

FIG. 33 shows final tightening of the spinal stabilization systems.

### DETAILED DESCRIPTION

The present application relates to minimally invasive devices and methods for assisting in the delivery of fixation devices and other implants to a target location in a patient. While the minimally invasive devices described herein can be used to assist various procedures, in some embodiments, they are used to assist in delivering fixation devices and other implants to help stabilize the spine. For example, a spinal stabilization system (e.g., including a bone fastening assembly such as a pedicle screw and an elongated connecting member or a rod) can be used to provide stability to two or more adjacent vertebrae. The bone fastener assembly is placed in each of the vertebrae to be stabilized, and the elongated connecting member or rod is coupled to the assemblies.

### Spinal Stabilization System

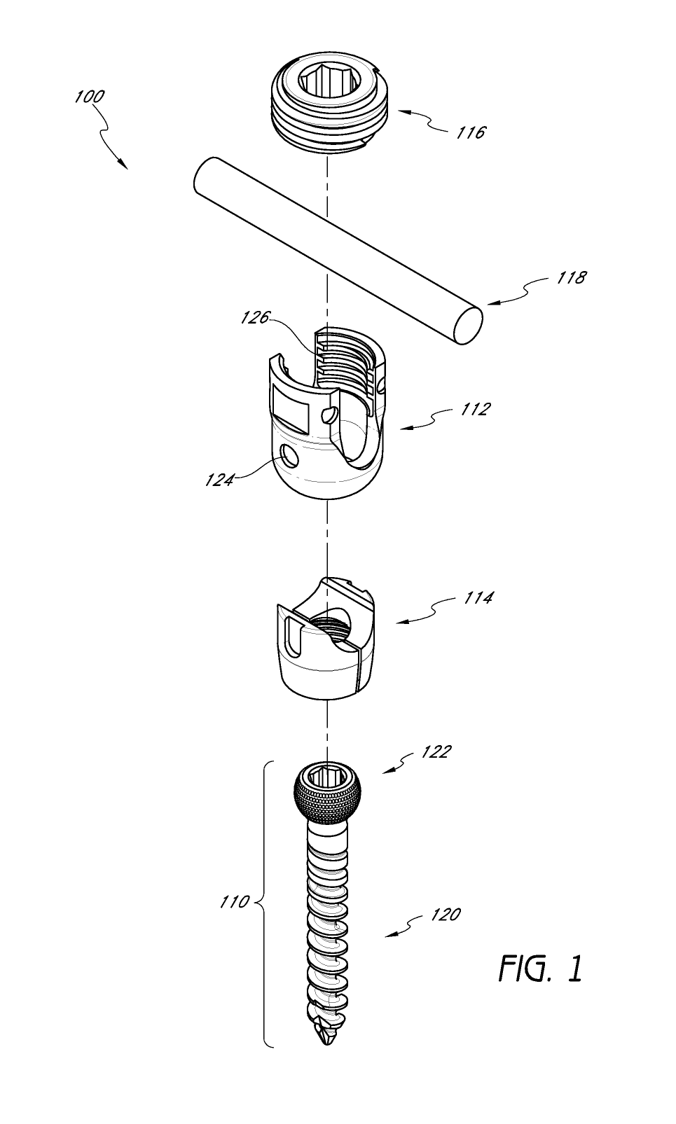

As shown in FIG. 1, an example embodiment of a spinal stabilization system 100 can include a screw 110, a housing 112, a set screw 116, and a rod 118. The system 100 can further include one or more clamps 114. The screw 110 can have a threaded shaft 120 configured to be secured to a vertebra and an enlarged semi-spherical head 122. The screw 110 can be cannulated and configured to receive a guidewire or k-wire as discussed herein. In some embodiments, the housing 112 can be tulip-shaped and can have a U-shaped seat for receiving the rod 118. The housing 112 can also include apertures or holes 124 on its outer surface for engaging a surgical access device as described in greater detail herein. The housing 112 can further include internal threads 126 at its upper end configured to receive the externally threaded set screw 116.

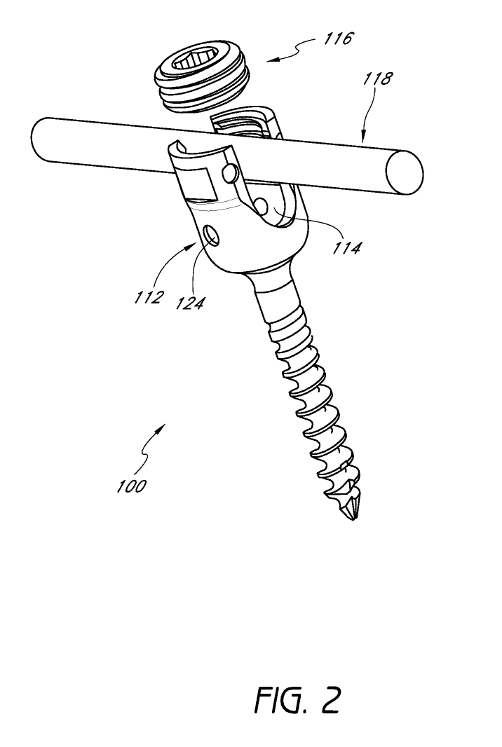

To assemble the system 100, the screw 110 and one or more clamps 114, if present, are loaded into the housing 112. When the system 100 is assembled, as shown in FIG. 2, the threaded shaft 120 extends through a lower opening in the housing 112, with the enlarged head sitting adjacent the lower opening. In some embodiments, the enlarged head sits directly on a lower internal surface of the housing. An inner surface of the one or more clamps 114 engages the enlarged head 122 of the screw 110, and an outer surface of the one or more clamps 114 engages an inner surface of the housing 112. Upper surfaces of the one or more clamps 114 are concave in shape and may act as a rod receiving surface to engage the rod 118. In some embodiments, the screw 110 can polyaxially rotate relative to the housing 112 prior to being fixed. Once the screw 110 has been inserted into the patient's vertebra, the rod 118 is inserted in the U-shaped seat of the housing 112 and engages an upper surface of the one or more clamps 114, if present. The set screw 116 is inserted into the housing 112 to secure the rod 118 and fix the screw 110 relative to the housing 112.

Additional details regarding example spinal screws are described in U.S. Patent Publication No. 2010/0241175, filed Jul. 28, 2009, entitled “Pedicle Screws and Methods of Using the Same,” the entirety of which is hereby incorporated by reference. Other types of screws, including various pedicle screws, can also be used with the devices and methods described herein. It will be appreciated that the clamps 114 can be replaced with a washer or other intervening member which may have a concave rod receiving surface configured to receive the rod as described above.

### Minimally Invasive Tower Access Device

In some embodiments, a minimally invasive tower access device is provided. The access device includes an outer sleeve and an inner sleeve that telescopingly or slidably engage with one another. The inner sleeve includes one or more grasping elements that can grasp a fixation device (e.g., a spinal screw such as a pedicle screw) for delivery into a bone member of a spine. Once the access device is coupled to the spinal screw, the access device and spinal screw can be delivered either through an incision in an open surgery, or minimally invasively through a relatively smaller incision, such as percutaneously. Once through the incision, the spinal screw can be brought to a location proximate to a bone member where it can be inserted.

The access device can serve as a portal or opening that extends from the bone member to outside of the patient. Instruments can be delivered through the access device. For example, a screw delivery device can be provided through the access device to secure the spinal screw to the bone member. A number of additional instruments can be used with the access device to provide spinal stabilization, for example, a rod inserter, a threaded rod reducer, and a set screw inserter. In addition, implants can be delivered adjacent the side of the access device. For example, a rod implant can be delivered along the side of the access device which can connect in between the implanted screws. By using one or more access devices to deliver screws or other implants as described herein, a spinal stabilization system can be formed. The one or more access devices advantageously allow screws and other implants to be inserted in a specific location with ease, and allow for a surgeon to comfortably maintain external control of the screw from outside of a patient's body.

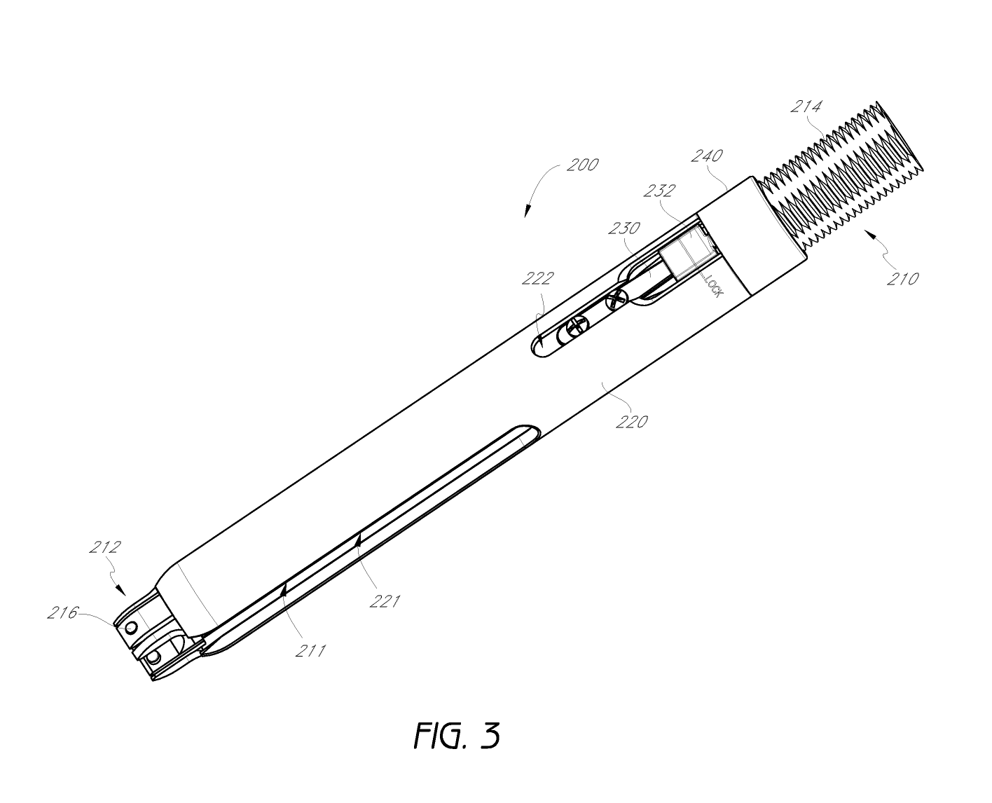

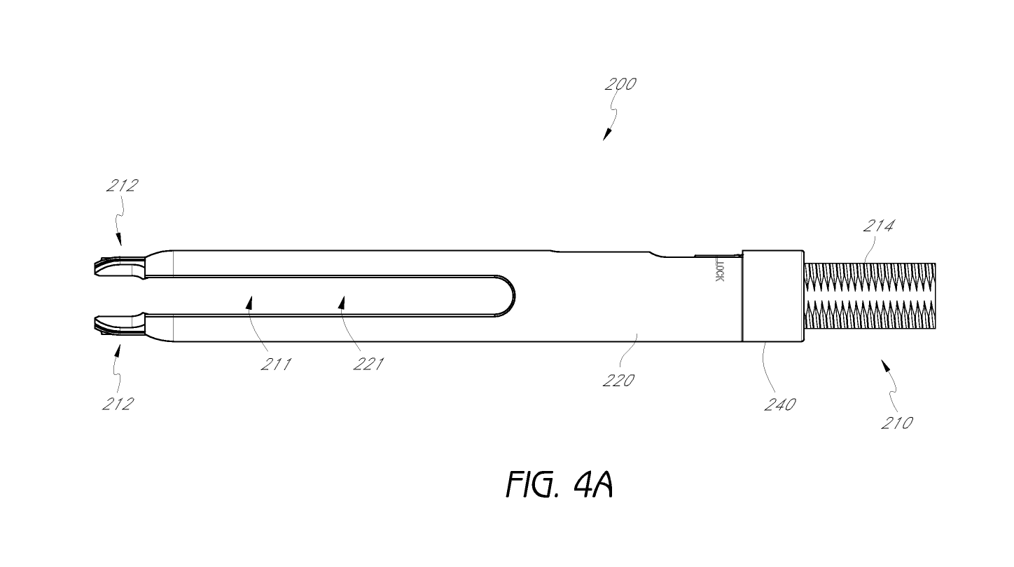

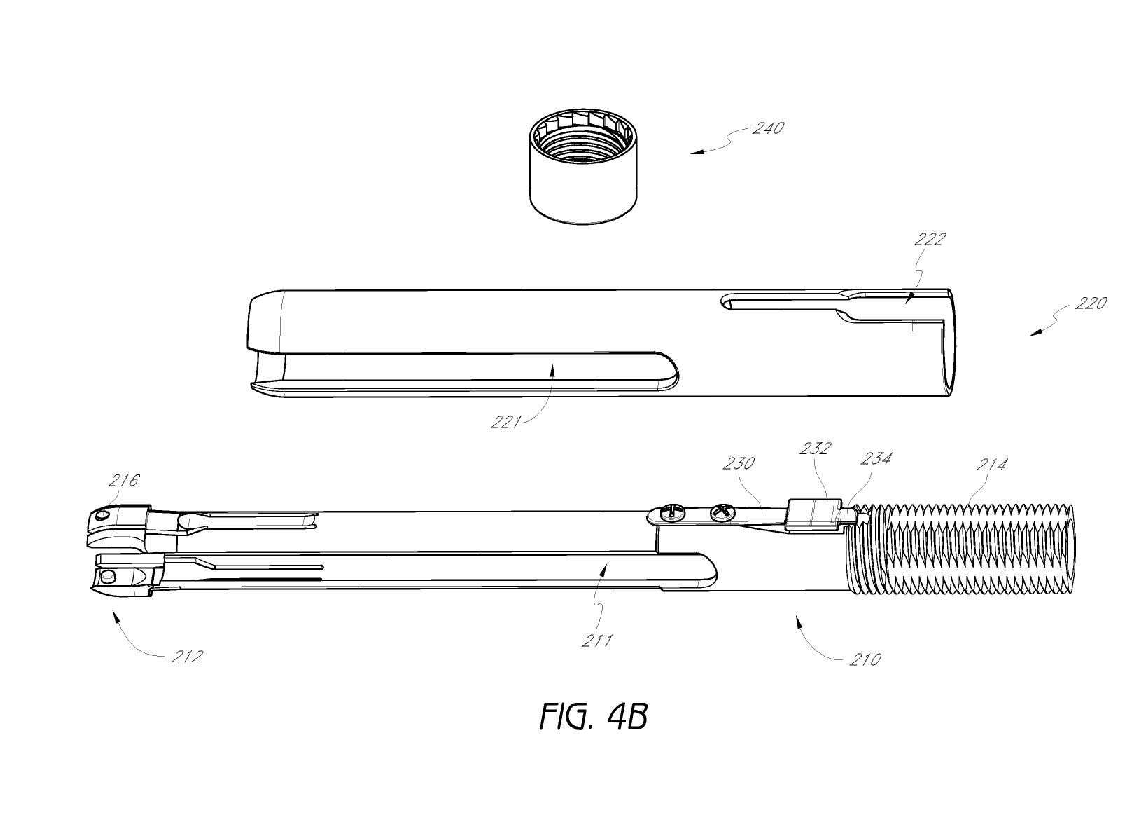

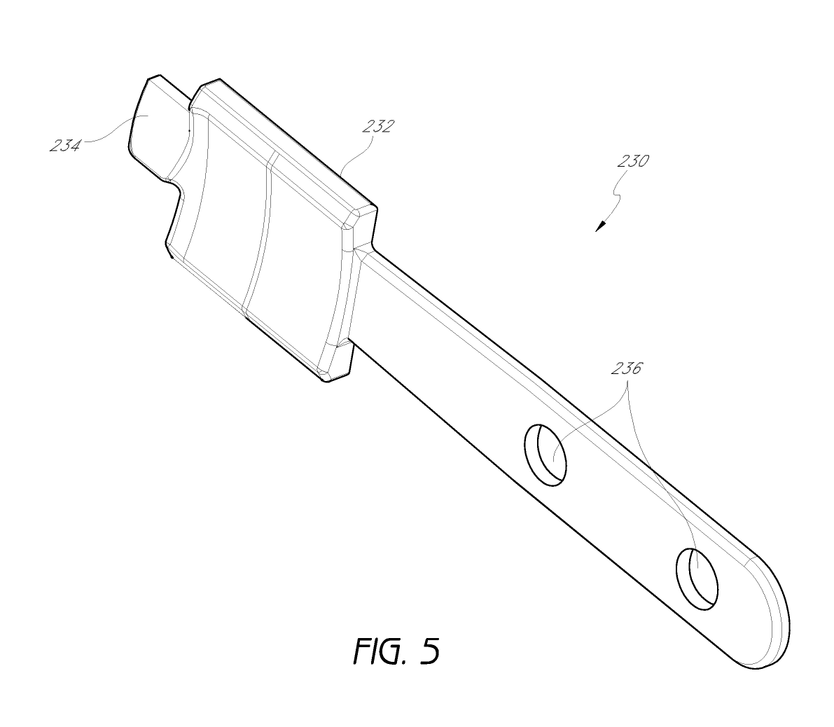

An example embodiment of a minimally invasive tower access device 200 is shown in FIGS. 3 and 4A. As shown, the access device 200 includes an elongated outer sleeve 220 that slidably receives an elongated inner sleeve 210. The inner sleeve 210 includes a spring latch 230, shown in FIG. 5. A lock nut 240 engages a portion of the inner sleeve 210. When all of the components are assembled as shown in FIGS. 3 and 4A, they form an access device 200 that can be used to deliver fixation devices, for example, a spinal stabilization system 100 as shown and described herein, to a target location of a patient's spine via minimally invasive procedures.

FIG. 4B illustrates an exploded view of various components (e.g., the outer sleeve, inner sleeve, and lock nut) of the tower access device of FIGS. 3 and 4A. The outer sleeve 220 is a hollow cylindrical body extending from a proximal end (i.e., the end exposed during surgery) to a distal end (i.e., the end inside the patient's body during surgery) and is configured to receive the inner sleeve 210 therein. As shown, the outer sleeve 220 includes three slots, a proximal slot 222 formed on the proximal end of the outer sleeve 220 and a pair of distal slots 221 formed on the distal end of the outer sleeve 220. In other embodiments, the outer sleeve 220 can include a single distal slot 221. In the illustrated embodiment, the proximal slot 222 and distal slots 221 extend to the proximal and distal ends, respectively, of the outer sleeve 220, and partially along the length of the outer sleeve 220. The pair of distal slots 221 are located on opposite sides of the outer sleeve 220 In other embodiments, the proximal 222 and/or distal 221 slot can be formed within the body of the outer sleeve 220 rather than extending to the proximal or distal end.

In some embodiments, the proximal slot 222 opens along one side of the outer sleeve 220, while the distal slots 221 opens along two sides of the outer sleeve 220 (as shown in FIGS. 3-4B). The proximal slot 222 can be located in between the two openings that form the distal slots 221. While a center longitudinal axis of the proximal slot 222 is shown at about 90 degrees away from a center longitudinal axis of either of the distal slots 221 (as measured along the circumference of the outer sleeve 220), the proximal slot 222 can be located at any angle relative to the openings of the distal slots 221, such as between 0 and 90 degrees. While the proximal slot 222 is smaller in both width and length than the distal slots 221 in the illustrated embodiment, the slots need not be limited to these relative dimensions.

Both the proximal slot 222 and the distal slots 221 of the outer sleeve 220 can serve particular functions. In some embodiments, the proximal slot 222 can serve to receive the spring latch 230, which can be coupled to the inner sleeve 210. The proximal slot 222 can work in conjunction with the spring latch 230 to identify the current mode of operation of the access device 200 (e.g., “locked” or “unlocked” mode) as best shown in FIG. 3. The spring latch 230 can include a marker that can help identify the particular mode of operation. With the spring latch 230, the mode of operation of the access device 200 can be easily visible to the surgeon. In some embodiments, the distal slots 221 can serve to receive one or more stabilization implants therethrough. For example, in some embodiments, a stabilizing rod member, such as rod 118, can be delivered along the side of the access device 220 and angled through one or both of the distal slots 221.

The distal slots 221 of the outer sleeve 220 can have a length between 4 cm and 8 cm, or a length between 6 cm and 7 cm. In some embodiments, the lengths of the distal slots 221 are much longer (e.g., at least 5.5 cm) than slots in conventional access devices. In some embodiments, the lengths of the distal slots 221 of the outer sleeve 220 are between ⅓ and ¾, or approximately ½ in some instances, the length of the entire body of the outer sleeve. In some embodiments the distal slots 221 of the outer sleeve can be even longer, and can extend almost the entire length of the outer sleeve 220. A longer slot advantageously allows a rod implant can be more easily delivered through the slot to provide spinal stabilization. In addition, providing a longer slot length makes the instrument lighter by removing material from the system. A challenge, however, is that with the longer slot, the sidewalls that form the slot may need to be stronger in order to withstand forces on the sidewalls in some embodiments. Accordingly, in some embodiments, the thickness of the sidewalls that form the longer distal slots 221 of the outer sleeve 220 are preferably increased relative to conventional sleeves to withstand forces on the sidewalls. In some embodiments, the thickness of the sidewalls that form the longer distal slots 221 are between about 0.05 cm and 0.4 cm, or between about 0.2 cm and 0.3 cm.

As shown in FIG. 4B, the inner sleeve 210 is also a hollow cylindrical body extending from a proximal end to a distal end. The inner sleeve 210 can be slidably received within the outer sleeve 220, and in some embodiments, can be secured in a position relative to the outer sleeve 220 by using the lock nut 240. In some embodiments, a proximal end of the inner sleeve 210 includes an externally threaded portion 214, and the lock nut 240 includes internal threads configured to threadingly engage the threaded portion 214. In some embodiments, the inner sleeve 210 has an interior diameter of between about 0.5 cm and 2 cm, or between about 1.0 cm and 1.1 cm.

A distal section of the inner sleeve 210 includes distal slots 211 and a pair of compressible grasping elements 212. Like the distal slots 221 of the outer sleeve 220, the distal slots 211 of the inner sleeve 210 can open on two sides of the inner sleeve 210. In some embodiments, the distal slots 211 of the inner sleeve 210 are approximately the same size (e.g., similar width and height) of the distal slots 221 of the outer sleeve 220. One skilled in the art will appreciate that the dimensions of both the distal slots 221 of the outer sleeve 220 and slots 211 of the inner sleeve 210 can vary with respect to one another. The distal slots 211 of the inner sleeve 210 can be placed in part or in complete alignment with the distal slots 221 of the outer sleeve 220. In some embodiments, when the distal slots 211 of the inner sleeve 210 is aligned with the distal slots 221 of the outer sleeve 220, a rod implant that is delivered into the patient can pass through both of the slots. The rod implant can be angled through the slots such that each end of the rod implant makes contact with a screw head within the access device 200.

The distal slots 211 of the inner sleeve 210 can have a length between about 4.0 cm and 8.0 cm, or between about 6.0 cm and 7.0 cm. In some embodiments, the length of the distal slots 211 is much longer (e.g., at least 5.5 cm) than slots in conventional access devices. In some embodiments, the length of the distal slots 211 of the inner sleeve 210 is between ⅓ and ¾, or approximately ½ in some instances, the length of a non-threaded body of the inner sleeve 210.

In some embodiments, the pair of grasping elements 212 comprises a pair of compressible arms or tines for receiving a portion of a spinal stabilization system, such as housing 112 described herein. One skilled in the art will appreciate that the shape of the grasping elements 212 need not be limited to the description described herein. In some embodiments, the distance from one grasping element to another is slightly greater than the diameter of the hollow interior of the outer sleeve 220 in an uncompressed state. In these embodiments, in order for the inner sleeve 210 to be received through the proximal end of the outer sleeve 220, the grasping elements 212 should be slightly compressed. When the grasping elements 212 exit the distal end of the outer sleeve 220, the grasping elements 212 can return to their uncompressed state, thereby advantageously helping to secure the inner sleeve 220 to the outer sleeve 210 by limiting the inner sleeve 220 from unintentionally backing out of the outer sleeve 210.

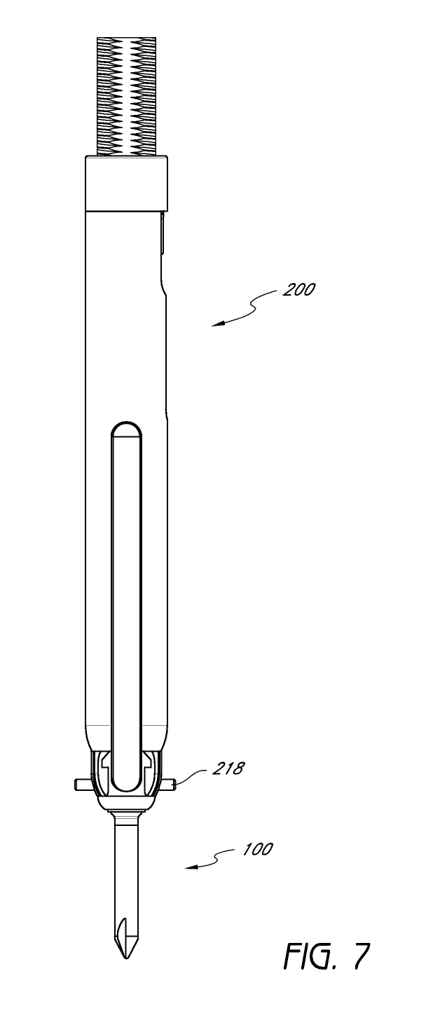

In some embodiments, the grasping elements 212 are flat, while in other embodiments (as shown in FIG. 4B), the grasping elements 212 can include some curvature so as to accommodate a screw head of a particular shape. In some embodiments, the grasping elements 212 include protruding members 216 that can be received in apertures 124 of the housing 112 to secure the housing 112 to the inner sleeve 210. The protruding members 216 can be rigid or somewhat flexible, and are configured to be inserted into two or more holes or apertures 124 formed on the housing 112 upon compression of the grasping elements 212 of the inner sleeve 210. While the protruding members 216 can have a smooth surface finish, in some embodiments, the protruding members 216 have a roughened surface finish that can provide a frictional force between the protruding members 216 and surfaces of the housing 112 that form the receiving apertures 124. The protruding members 216 can have a cross-sectional area that is circular, rectangular, trapezoidal or any other shape, so long as they are securely receivable in a corresponding aperture of the housing 112. In some embodiments, in addition to or as an alternative to the protruding members 216 configured to be received in apertures 124 of the housing 112, the grasping elements 212 include one ore more pins 218 configured to be received in one or more holes 124 in the housing 112, for example as shown in FIG. 7. In some embodiments, rather than have protruding members that resemble pins, the inner sleeve 210 can include flanges that extend from a bottom surface of the distal end of the inner sleeve 210. The flanges can be compressible such that when compressed, the flanges surround and secure a portion of the housing 112 (such as a bottom portion), thereby coupling the inner sleeve 210 to the housing 112.

The inner sleeve 210 can be slidably received in the outer sleeve 220 such that the grasping elements 212 of the inner sleeve 210 can extend beyond the distal end of the outer sleeve 220. In some embodiments, the inner sleeve 210 can be slidably received in the outer sleeve 220 such that in a first position, the grasping elements 212 are uncompressed. Sliding the inner sleeve 210 relative to the outer sleeve 220 in a second position can result in compression of the grasping elements 212. For example, the outer sleeve 220 can be slid down the inner sleeve 210 such that the distal end of the outer sleeve 220 helps to compress the grasping elements 212. In some embodiments, the outer sleeve 220 can completely cover the grasping elements 212 to compress the grasping elements, while in other embodiments, the outer sleeve 220 only covers a portion of the grasping elements 212 to cause compression. The compression mechanism provided by the outer sleeve 220 sliding over the compressible grasping elements 212 of the inner sleeve 210 is advantageous over conventional screw delivery devices, as the body of the outer sleeve 220 helps to reduce the risk of the protruding members 212 becoming accidentally loose from the housing 112. Moreover, having a slidably engaged outer sleeve 220 and inner sleeve 210 reduces the need for extra tools that might be used in conventional screw delivery devices for securing an access device to a screw member.

The inner sleeve 210 can also include a spring latch member 230. The spring latch 230 can include a button 232 and tab member 234 as shown in FIG. 5. In the illustrated embodiment, the tab member 234 is shaped as a ramp having a shoulder of about 90 degrees. The button 232 may include the marker described above for indicating a locked or unlocked position. The spring latch 230 can also include holes 236 for receiving one or more fixation members (e.g., screws) for attaching the spring latch 230 to the inner sleeve 210. As illustrated in FIG. 4B, the spring latch is fixed to the inner sleeve at or near a distal end of the spring latch with a pair of screws. At the proximal end of the spring latch, the button 232 and the tab member 234 are moveable between an elevated position with respect to the outer wall of the inner sleeve 210, and a depressed position where the button 232 and tab 234 come closer to the outer wall. This movement may be accommodated by providing a recess in the outer wall where the proximal end of the spring latch is located. The tab member 234 extends at least partially into the threaded portion 214. The button 232 can be sized and shaped to be easily depressed by a user using a thumb or other finger.

The spring latch 230 can serve multiple functions. In some embodiments, the spring latch 230 (when fixed to the inner sleeve 210) can fit within the proximal slot 222 of the outer sleeve 220 and can serve to identify the current mode of operation of the access device 220 when the inner sleeve 210 and outer sleeve 220 are slid relative to one another. For example, the spring latch 230 can include a marker on the button 232 that can identify when the outer sleeve and inner sleeve are in an “unlocked” position in which the two sleeves remain slidable relative to one another. Additionally, alignment of the proximal slot 222 with the spring latch 230 can advantageously promote proper placement of the outer sleeve 220 relative to the inner sleeve 210 so that the distal slot 221 of the outer sleeve 221 aligns with the distal slot 211 of the inner sleeve 210.

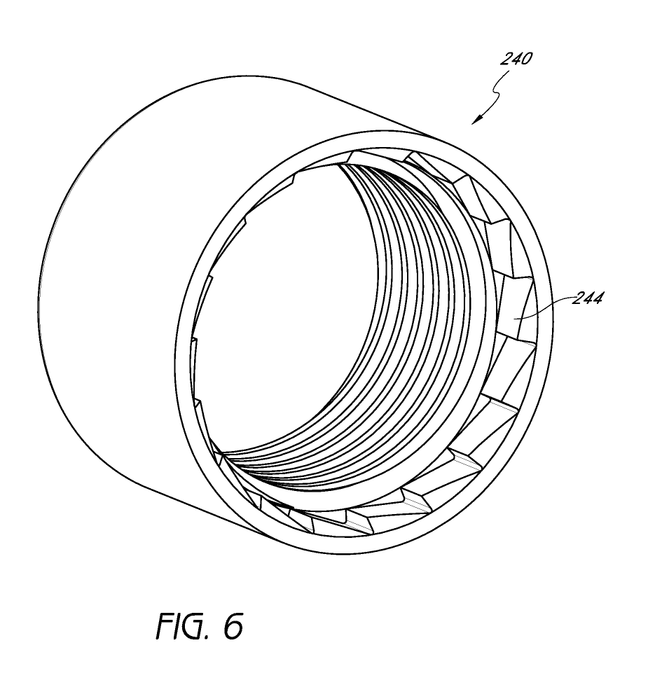

In some embodiments, the tab member 234 of the spring latch can interact with lock nut 240 when the lock nut 240 is rotated to a distal section of the external threaded portion 214 of the inner sleeve 210. For example, the tab member 234 can interact with an inner engagement surface 244 of the lock nut 240 to advantageously limit unintentional counter or back rotation of the lock nut 240 when in use. As discussed above, the button 232 and tab member 234 of the spring latch are moveable between an elevated position and a depressed position. In the elevated position, the tab member 234 engages the inner engagement surface 244 of the lock nut 240. In the depressed position, the tab member 234 releases from the inner engagement surface 244. In some embodiments, for example as shown in FIG. 6, the inner engagement surface 244 of the lock nut 240 is shaped as a ramp having a shoulder of about 90 degrees to correspond to the shape of the tab member 234.

In use, the lock nut 240 is used to secure the inner sleeve 210 and outer sleeve 220 in a locked mode by threading the lock nut 240 onto the threaded portion 214 of the inner sleeve 210 until the inner engagement surface 244 engages the tab member 234. The lock nut 240 engages the proximal end of the outer sleeve, such that further rotation of the lock nut 240 moves the inner sleeve proximally relative to the outer sleeve. This causes compression of the grasping elements. In the locked mode, the inner sleeve 210 is secured in position relative to the outer sleeve 220, and the grasping elements 212 of the inner sleeve 210 are compressed about a fixation device, for example as shown in FIG. 7. The two sleeves are locked together because the interaction between the inner engagement surface 244 and tab member 234 advantageously limits unintentional counter or back rotation of the lock nut 240 in use. To release the lock nut 240, the surgeon or other user depresses the button 232 which disengages the tab member 234 from the ramps of the inner engagement surface 244. This permits counter-rotation of the lock nut to permit distal movement of the inner sleeve relative to the outer sleeve. When the ramps of the lock nut and the spring latch tab are not engaged, the inner and outer sleeves are moveable relative to one another to permit compression and expansion of the grasping elements.

Additional details regarding example tower access devices having features that may be incorporated in the devices above, and their methods of manufacture and use, can be found in U.S. Patent Publication No. 2012/0022594, filed Jul. 26, 2010, entitled “Minimally Invasive Surgical Tower Access Devices and Related Methods,” the entirety of which is hereby incorporated by reference.

### Dilation Tubes

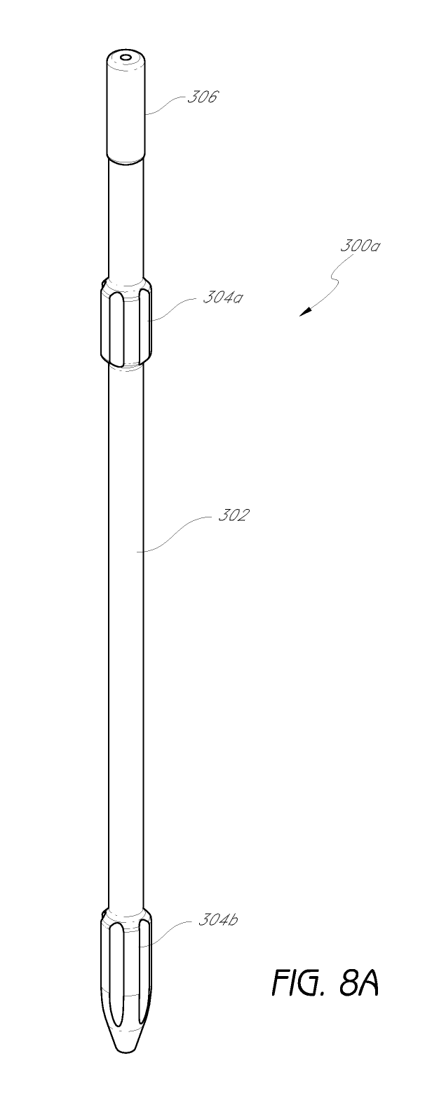

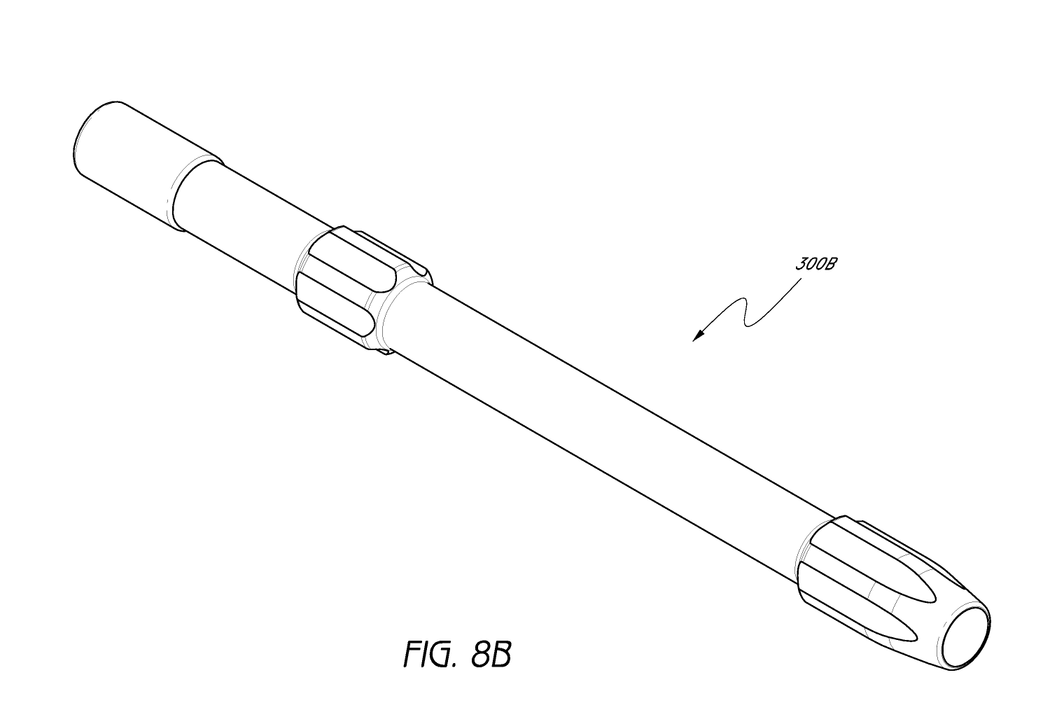

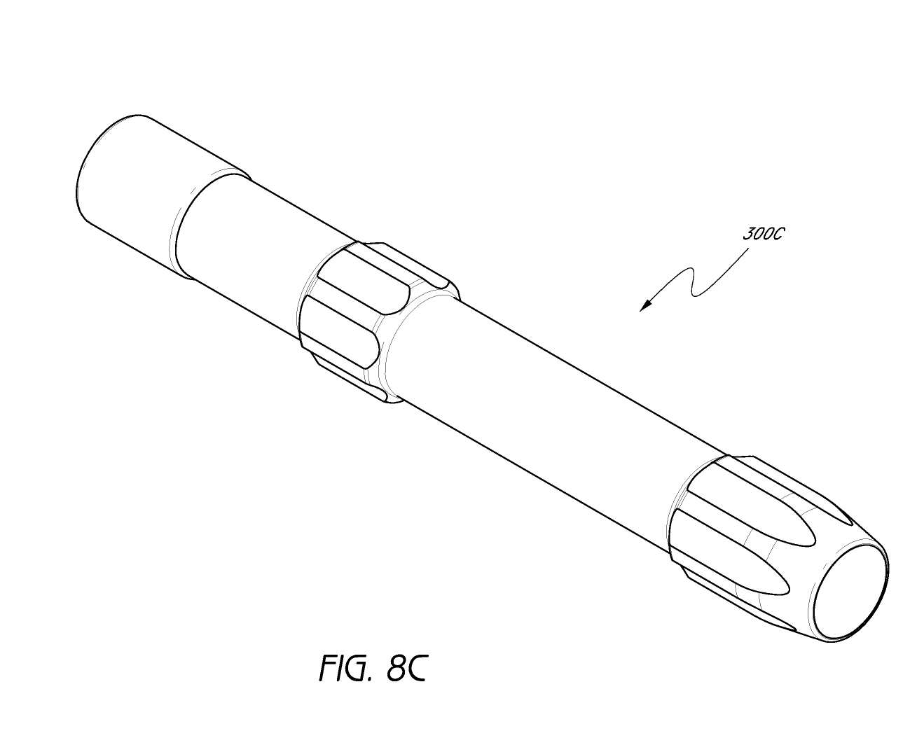

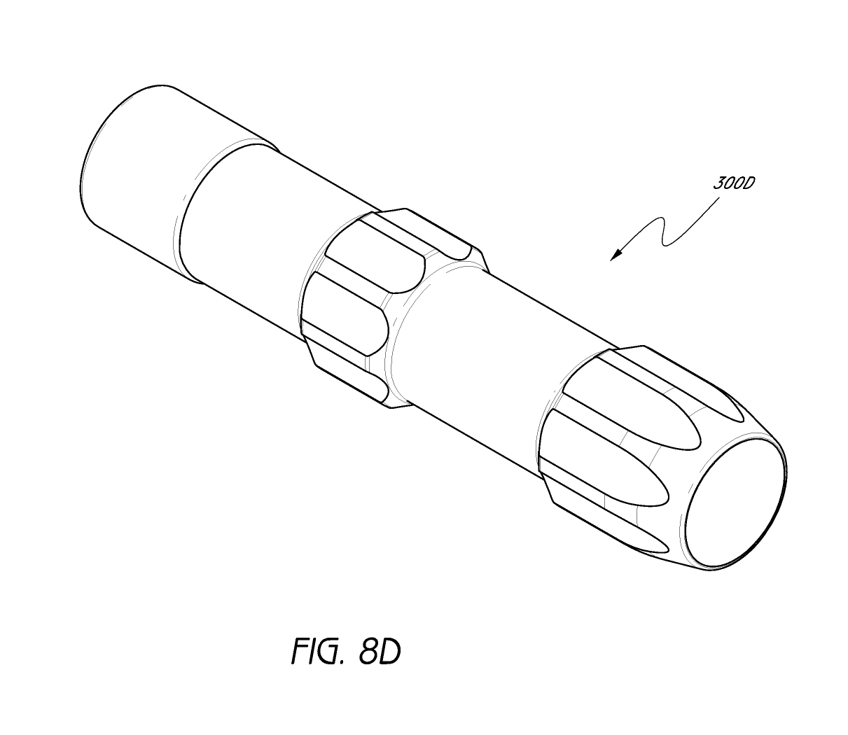

Various instruments can be used to prepare an insertion site in a patient's spine for implants such as spinal stabilization system 100 as described herein. For example, minimally invasive surgical procedures often make use of dilators to gradually enlarge a working channel. FIGS. 8A-8D show example embodiments of improved dilation tubes 300 having increasing dimensions. FIGS. 8A-8D show an 8mm dilation tube 300a, 13 mm dilation tube 300b, 18 mm dilation tube 300c, and 24 mm dilation tube 300d, respectively. The dilation tubes 300 include a shaft 302 and two load bearing portions 304a, b. In some embodiments, the load bearing portions 304a, b are integrally formed with the shaft 302. A dilation tube 300 can be made by starting with a hollow cylindrical tube having an inner diameter that is the desired inner diameter of the final tube 300 and an outer diameter approximately the same or greater than the final outer diameter desired for the load bearing portions 304a, b. The tube can then be cut, shaved, or otherwise reduced in diameter to achieve the final desired dimensions. Alternatively, in some embodiments, the load bearing portions 304a, b are collars removably or permanently coupled to the shaft 302. In some embodiments, the load bearing portions 304a, b are contoured, for example as shown in FIGS. 8A-8D, to enhance the user's grip. As illustrated, the load bearing portions 304a, 304b may have a plurality of parallel, longitudinal grooves extending along their lengths. In the illustrated embodiment, a distal end of the distalmost load bearing portion 304b transitions to a more narrow distal tip, which can facilitate insertion of the dilation tube into the working channel.

The shaft 302 has a smaller outer diameter than the load bearing portions 304a, b to advantageously reduce the overall weight of the dilation tube. For example, the 8 mm dilation tube can have a length of about 7.550 inches (in.), an inner diameter of about 0.069 in., an outer shaft 302 diameter of about 0.229 in., and an outer load bearing portion 304a, b diameter of about 0.335 in. The 13 mm dilation tube can have a length of about 6.550 in., an inner diameter of about 0.345 in., an outer shaft diameter of about 0.405 in., and an outer load bearing portion diameter of about 0.539 in. The 18 mm dilation tube can have a length of about 5.550 in., an inner diameter of about 0.549 in., an outer shaft diameter of about 0.609 in., and an outer load bearing portion diameter of about 0.740 in. The 24 mm dilation tube can have a length of about 4.550 in., an inner diameter of about 0.750 in., an outer shaft diameter of about 0.810 in., and an outer load bearing portion diameter of about 0.975 in. The dilation tubes can also have an enlarged portion 306 at a proximal end. The enlarged proximal portion can have an outer diameter of about 0.249 in. for the 8mm dilation tube, about 0.445 in. for the 13 mm dilation tube, about 0.649 in. for the 18mm dilation tube, and about 0.850 in. for the 24 mm dilation tube. In some embodiments, the enlarged portion includes a knurled surface to improve grip.

### Awl and Tap

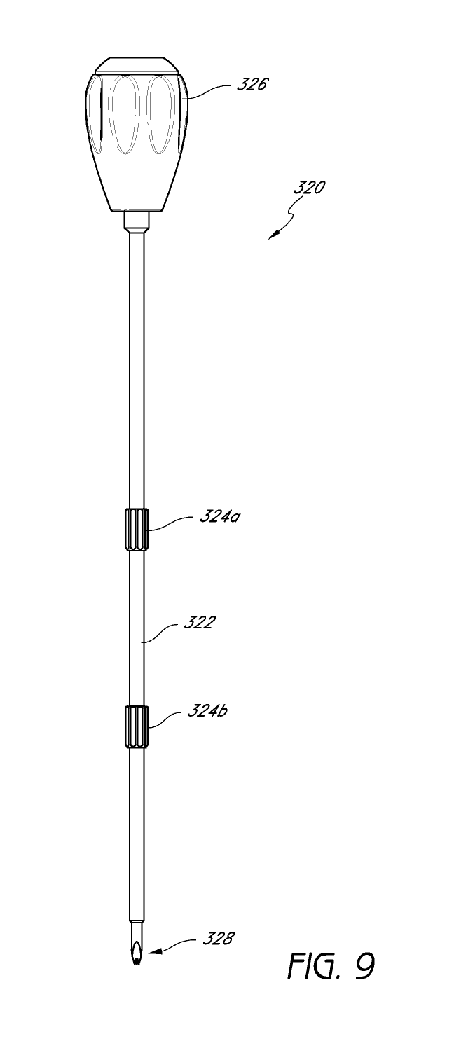

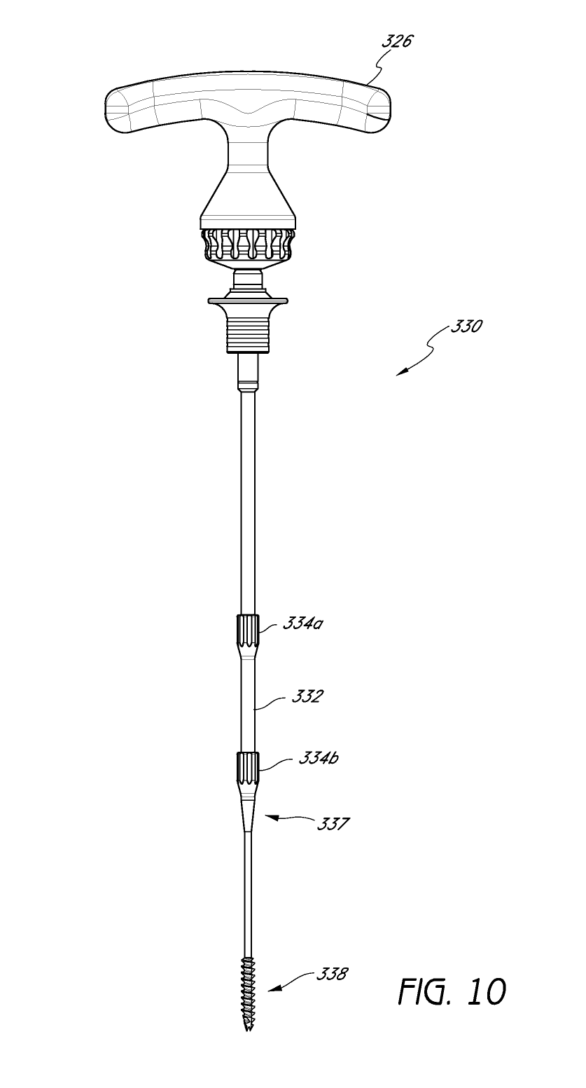

As shown in FIGS. 9 and 10, an improved awl 320 and tap 330 can each have two load bearing portions 324a, b, 334a, b and a reduced diameter shaft 322, 332 similar to the dilation tube 300 to advantageously reduce the weight of the instruments. The load bearing portions 324a, b, 334a, b of the awl 320 and tap 330 can also be integrally formed or coupled to the shaft 322, 332 as described herein with respect to dilation tube 300, and can be similarly contoured to improve grip.

As shown, a distal end of the awl 320 includes a trocar tip 328 having sharp edges to allow the surgeon or other user to create a pilot hole for an implant. A distal end of the tap 338 includes external threading having a cutting edge to create female threads in the pilot hole. A portion 337 of the awl shaft 332 distal and adjacent to the distalmost load bearing portion 334b can taper (i.e., decrease in diameter) toward the distal end so that the shaft 332 has a smaller diameter adjacent the distal end 338. The awl 320, tap 330, and/or other instruments described herein can include various types of handles. As illustrated, the awl 320 includes a palm handle 326 at a proximal end of the shaft 322, and the tap 330 includes a T-handle 336 at a proximal end of the shaft 332. In some embodiments, the T-handle 336 can be coupled to the shaft 322 via a hex adapter. Other types of handles (e.g., axial, trilobe, etc.) and coupling mechanisms are also possible.

### Screw and Rod Delivery Devices

Once the insertion site(s) have been prepared, the tower access device 200 or another access device can be coupled to a spinal stabilization system 100 or another implant and inserted into the working channel to the target site, as described further below. Various instruments according to the present disclosure can be used, alone or in combination with tower access device 200 or another access device, to deliver fixation devices and/or other implants, such as spinal stabilization system 100, to a patient's spine.

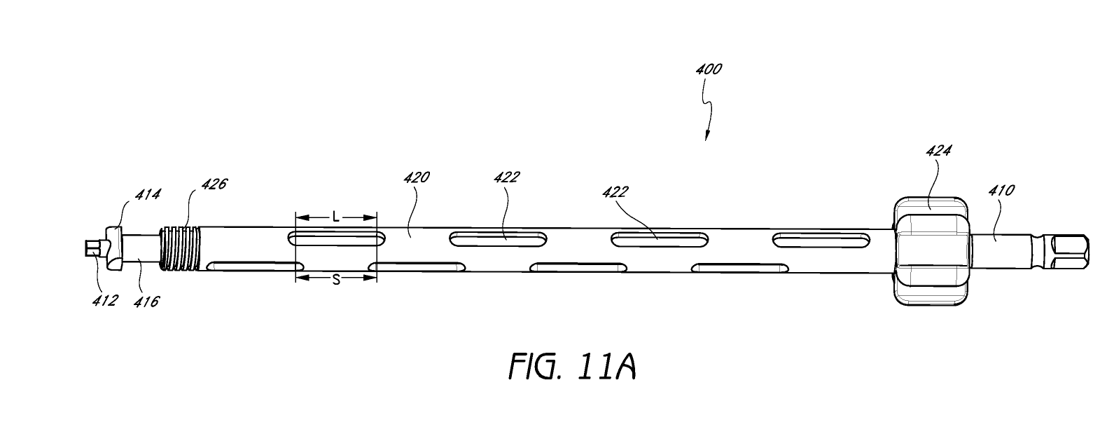

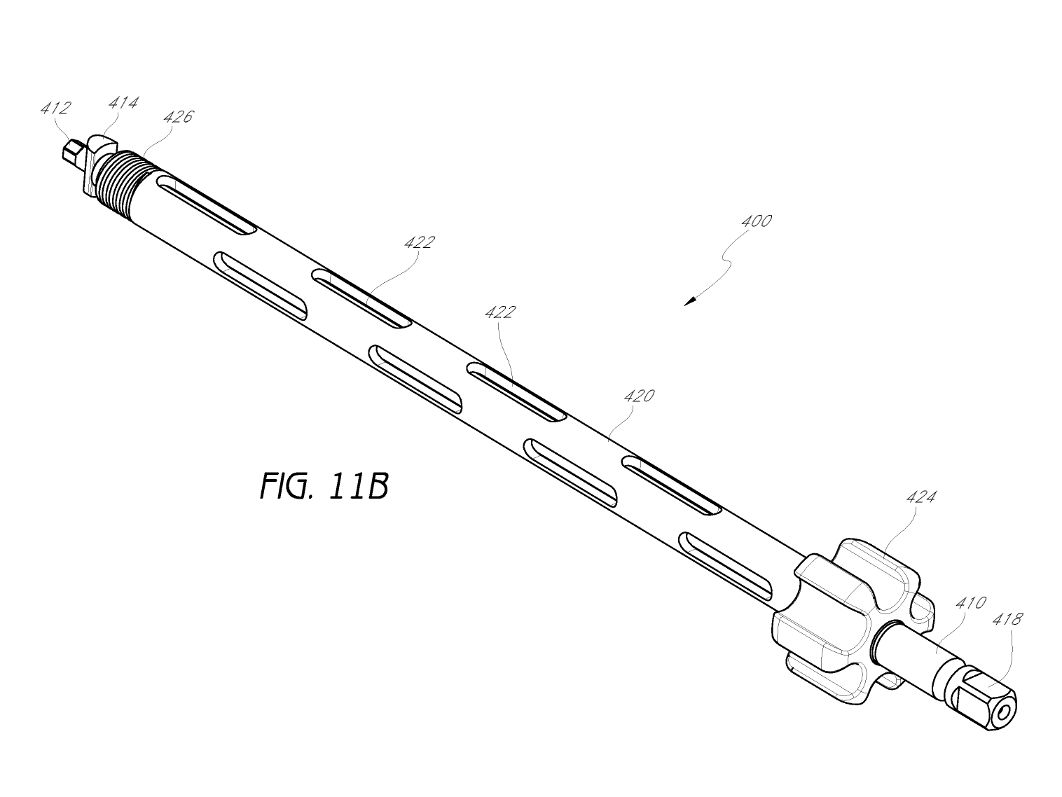

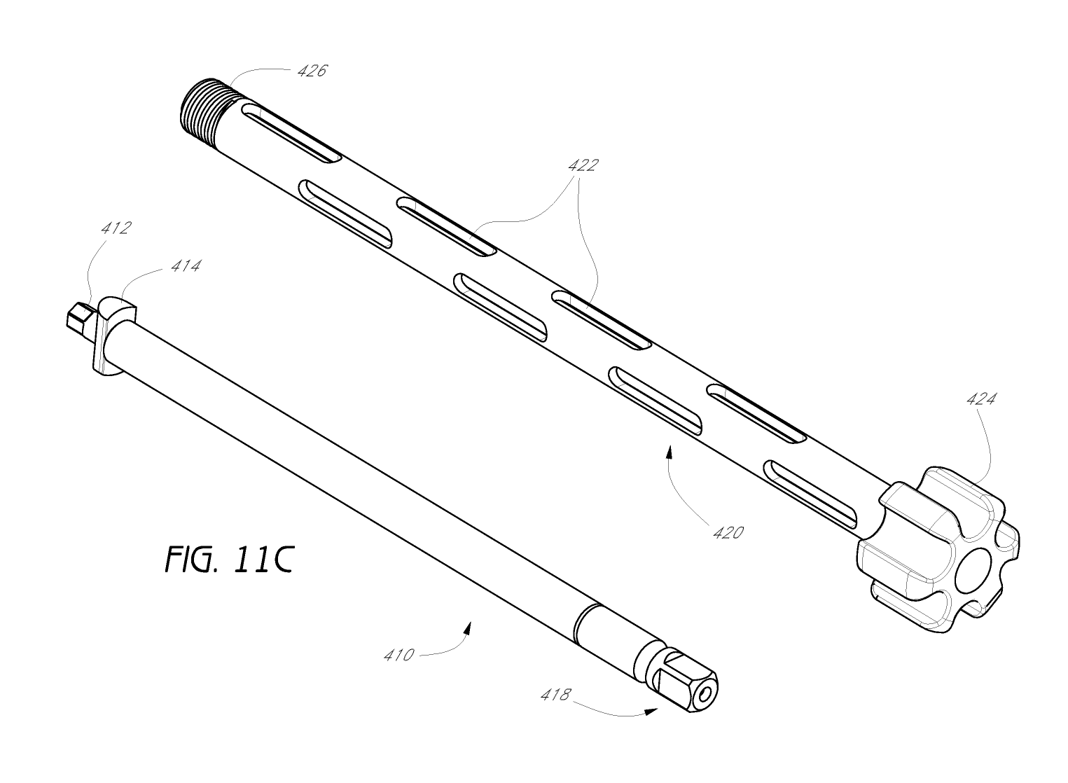

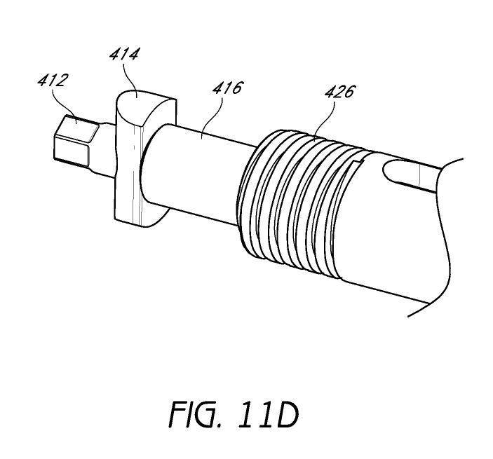

For example, screw delivery device 400 shown in FIGS. 11A-11D is configured to be inserted through the inner sleeve 210 of the access device 200. In some embodiments, screw delivery device 400 has inner 410 and outer 420 shafts that are axially and rotationally moveable relative to each other. In some embodiments, the inner 410 and/or outer 420 shafts have one or more slots 422 to advantageously reduce the weight of screw delivery device 400. In the illustrated embodiment, the outer shaft 420 includes four longitudinal rows of four slots 422 each, for a total of sixteen slots 422. Adjacent rows of slots 422 can be staggered as shown in FIG. 11A. The slots have a width of about 0.150 in. and a length L (as illustrated in FIG. 11A) of about 0.750 in. There is a spacing of about 0.750 in. between adjacent slots (measurement S illustrated in FIG. 11A). The total length of the outer shaft 420 can be about 7.000 in to about 7.020 in.

As shown in FIG. 11C, a distal end of the inner shaft 410 can have a hexalobe tip 412 to engage a corresponding recess on a bone screw, for example, screw 110. Other screw-screw delivery device engagement configurations are also possible. The inner shaft 410 can also include a saddle 414 near the distal end proximal to the hexalobe tip 412. The saddle 414 is configured to contact another portion of the implant, for example, the one or more clamps 114 or any other rod receiving surface of the bone fixation device in use. The saddle 414 can have a shape similar to a half cylinder to matingly engage the shape of the rod receiving surface. A proximal end 418 of the inner shaft 410 can be configured to be coupled to a handle, e.g., a palm, axial, tri-lobe, T, or other handle. In the illustrated embodiment, a proximal end of the outer shaft 420 includes a grip 424, and a distal end of the outer shaft 420 includes an externally threaded portion 426.

In use, the surgeon or other user inserts the screw delivery device 400 into the working channel, for example, through the tower access device 200, so that the hexalobe tip 412 engages the corresponding recess on the spinal screw and the saddle 414 contacts the rod receiving surface. The user can then use grip 424 to rotate the outer shaft 420 relative to the inner shaft 410 to thread the externally threaded portion 426 into the internal threads 126 of the housing 112 (shown in FIG. 1). As shown in FIGS. 11A and 11C, there may initially be a gap 416 between the saddle 414 and externally threaded portion 426. In some embodiments, the gap 416 closes as the externally threaded portion 426 of the outer shaft 420 is threaded down into the housing 112, for example as shown in FIG. 11B. In other embodiments; depending on the dimensions of the housing 112, a portion of the gap 416 may remain once the externally threaded portion 426 is threaded into the housing 112. The user can then rotate the inner 410 and outer 420 shafts together to insert the spinal screw 110 into the patient's spine. In some embodiments, the inner 410 and outer 420 shafts become substantially locked relative to one another by virtue of the hexalobe tip 412 engaging the recess on the spinal screw, the saddle 414 contacting the rod receiving surface, and the externally threaded portion 426 engaging the internal threads 126 of the housing 112 so that the inner 410 and outer 420 shafts rotate together when inserting the spinal screw 110 into the patient's spine. The spinal screw 110, rod receiving surface such as clamps 114, and housing 112 rotate together during insertion of the spinal screw 110 into the patient's spine. In some embodiments, rotation of the screw delivery device 400 causes rotation of the spinal stabilization system 100 as well as the access device 200 to which it is coupled. The saddle 414 contacting the one or more clamps 114 advantageously helps distribute the load during tightening of the spinal screw 110 to reduce the amount of torque on the hexalobe tip 412.

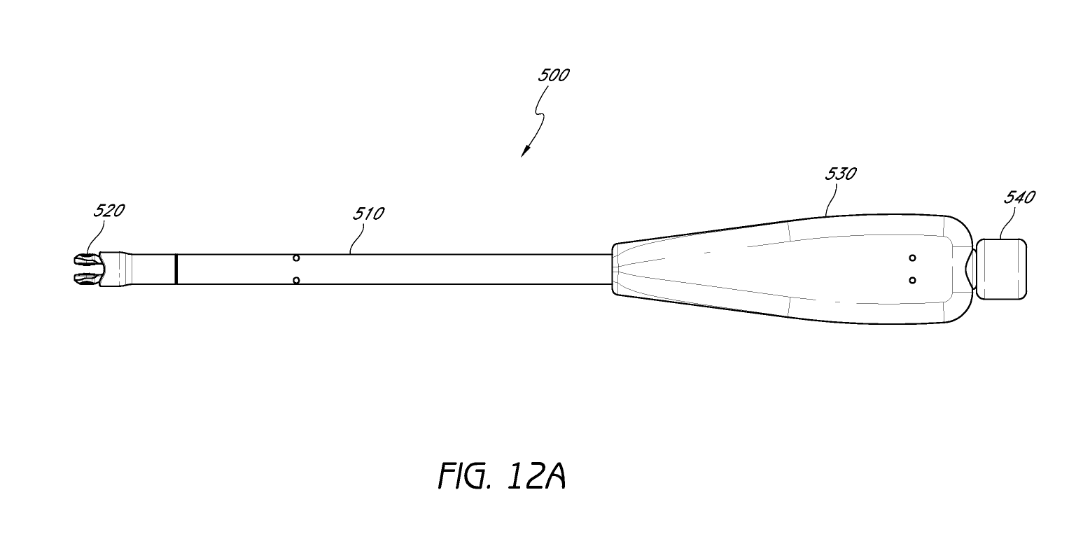

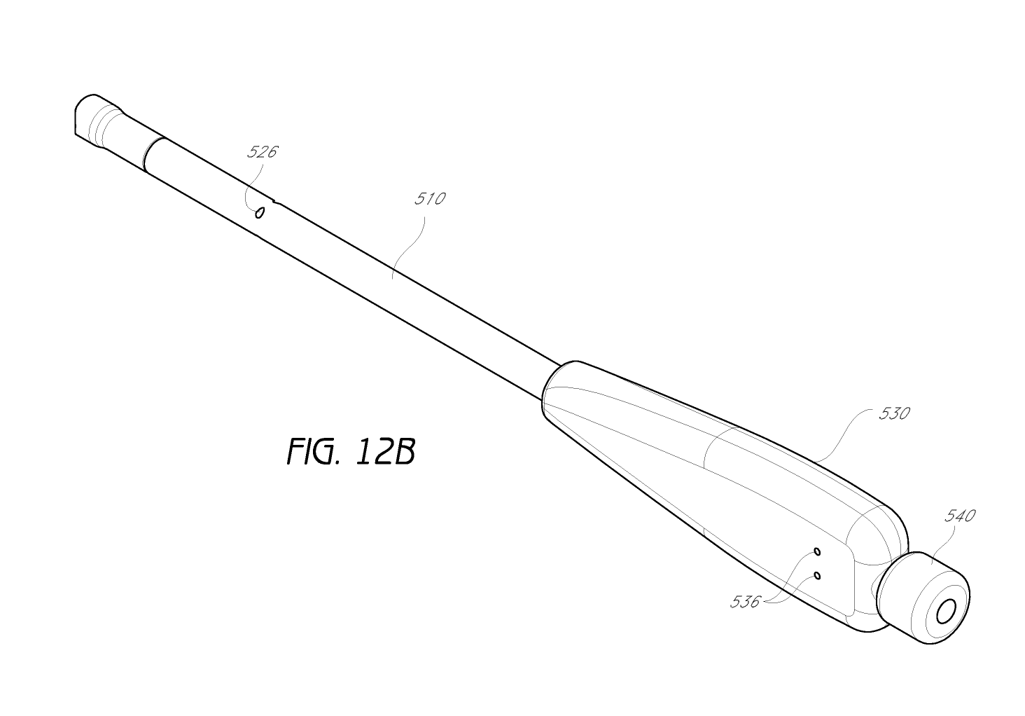

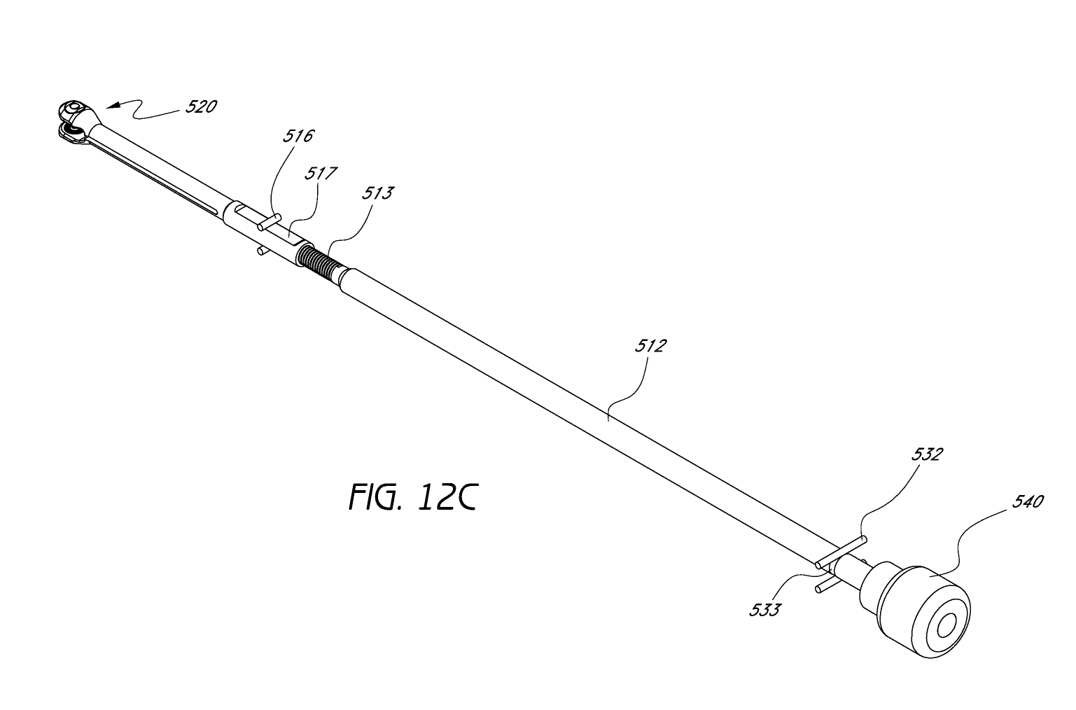

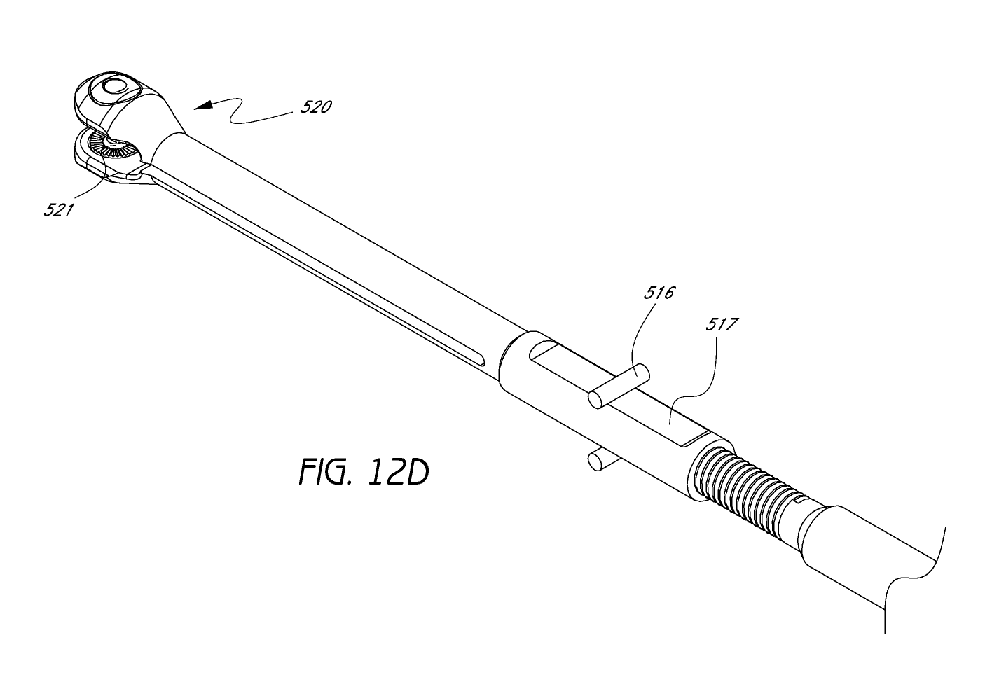

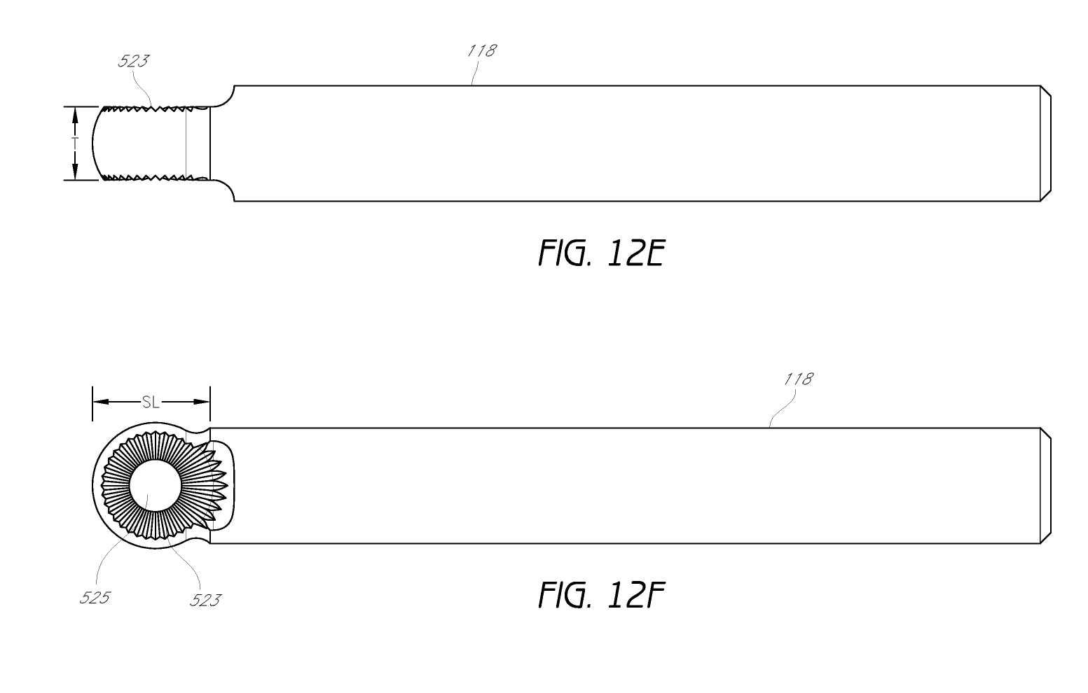

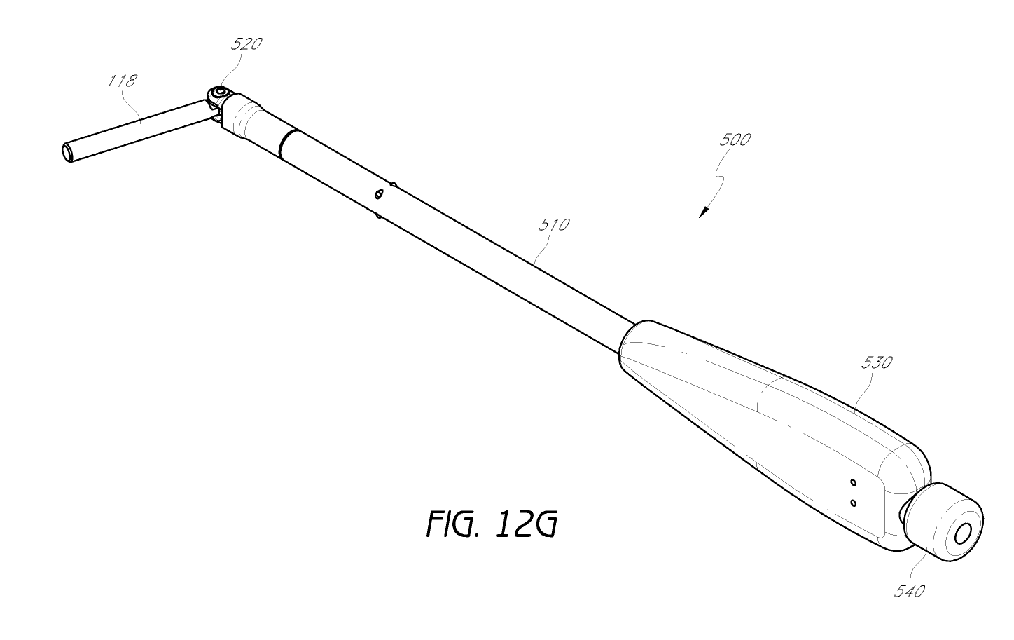

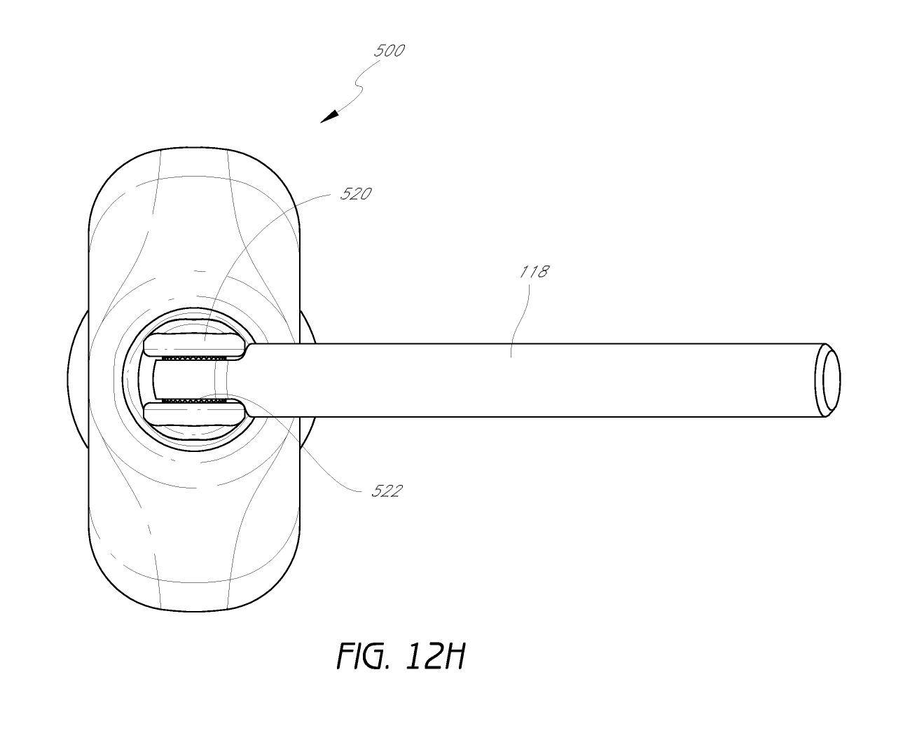

A rod insertion device 500 can be used to insert a spinal rod 118 into the U-shaped seat of housing 112. As shown in FIG. 12A, the rod insertion device 500 can include an elongate, generally cylindrical shaft 510 having a gripping end 520 at a distal end of the shaft 510 and a handle 530 at a proximal end of the shaft 510. The device 500 can further include an actuator knob 540. In the illustrated embodiment, the gripping end 520 includes two gripping elements configured to grip a rod 118, for example as shown in FIGS. 12G and 12H. In some embodiments, the gripping end 520 includes serrations 522 configured to mate with corresponding serrations 523 on the rod 118 as shown in FIG. 12C. In some embodiments, the rod 118 is substantially smooth over most of its length and has serrations over a portion of one or both ends. The serrated portion of the rod can also be flattened and more narrow than the remainder of the rod 118 as shown in FIGS. 12E and 12F. In the illustrated embodiment, the serrated portion has a thickness T of about 0.094 in. and a length SL of about 0.332 in. The serrations can advantageously provide a higher mechanical resistance to force as compared to a friction lock to enhance the grip of the gripping end 520 on the rod member 118. In some embodiments, each gripping element of the gripping end 520 includes a central pin 521 (shown in FIG. 12D) configured to engage a corresponding hole 525 through rod 118 (shown in FIG. 12F) at the end of the rod with the serrations.

The rod insertion device 500 can act as a collet to grip the rod 118. For example, the gripping end 520 can be coupled to an inner shaft 512 surrounded by shaft 510. As shown in FIG. 12B, the handle 530 is coupled to the proximal end of the shaft 510. As shown in FIG. 12C, the actuator knob 540 is coupled to the inner shaft 512. In some embodiments, the gripping end 520 is integrally formed with the inner shaft 512. Alternatively, the gripping end 520 can be coupled to the inner shaft 512 via a threaded connection 513 or any other suitable connection. As shown, the gripping end 520 can be coupled to the shaft 510 by two dowel pins 516. The pins 516 sit in troughs 517 on opposite sides of the gripping end 520 and engage the shaft 510 through a pair of openings 526 on either side of the shaft 510. The pins 516 can slide or roll in the troughs 517 to allow for relative movement between the shaft 510 and the gripping end 520 and inner shaft 512. In some embodiments, two additional dowel pins 532 couple the handle 530 to the shaft 510 and inner shaft 512. The pins 532 extend alongside the inner shaft 512, through the shaft 510 and engage the handle 530 through a pair of openings 536 on either side of the handle 530. Other arrangements of dowel pins and other coupling mechanisms are also possible.

In some embodiments, the shaft 510 can be advanced distally with respect to the inner shaft 512 to partially cover and compress the gripping end 520 so that the gripping end 520 tightens on the rod 118. In some embodiments, the inner shaft can be retracted proximally so that the gripping end 520 is partially pulled into shaft 510 to compress the gripping end 520. In some embodiments, the actuator knob 540 can be used to move the shaft 510 and the gripping end 520 relative to one another. The gripping end 520 can be locked and unlocked as needed during the procedure if the surgeon wants to adjust the angulation of rod insertion. In some embodiments, the surgeon can release the actuator knob 540 partway to release the grip of the serrations 522 on the rod 118 while pins on the gripping elements remain in place to hold the rod 118. The surgeon can then adjust the angle of the rod 118 and re-engage the actuator knob 540 to securely grip the rod 118.

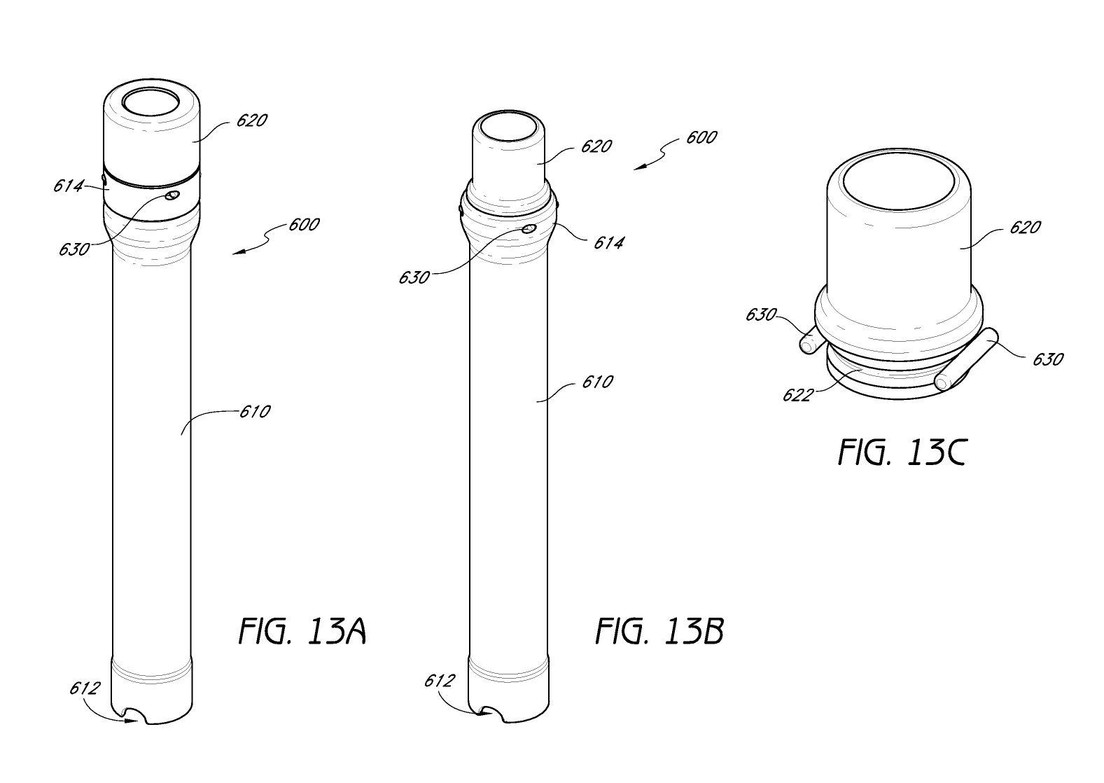

A threaded rod reducer 600, for example as shown in FIGS. 13A-13C, can be used to fully seat the rod 118 in the housing 112. In some embodiments, the rod reducer 600 is configured to be placed over the tower access device 200. The example threaded rod reducers 600 shown in the two different versions of FIGS. 13A and 13B include a cannula 610 and an internally threaded top portion 620. The threaded rod reducer 600 does not include a handle, which can advantageously reduce the size of the instrument and allow for easier operation during use. A distal end of the cannula 610 includes a slot 612 configured to engage the rod 118. In some embodiments, the slot 612 forms a half-circle having a radius of between about 0.1 cm and about 0.5 cm, or between about 0.3 cm and about 0.4 cm. At the distal end of the cannula 610, near the slot 612, the cannula 610 may have an enlarged diameter as compared to the rest of the cannula 610 body. A proximal end 614 of the cannula 610 can also have an enlarged diameter. The threaded top portion 620 is coupled to the proximal end 614 of the cannula 610. The proximal end 614 of the rod reducer of FIG. 13B has a contoured outer surface compared to the cylindrical proximal end 614 of the rod reducer of FIG. 13A. As shown in FIG. 13C, the threaded top portion 620 can include a reduced diameter portion 622 configured to sit within the proximal end 614 of the cannula 610. Two dowel pins 630 sit in the reduced diameter portion 622 and engage the proximal end 614 of the cannula 610 through a pair of openings on each side of the proximal end 614. The dowel pins 630 hold the top portion 620 and cannula 610 together while allowing the top portion 620 to rotate relative to the cannula 610.

In use, the threaded top portion 620 threads onto the proximal threaded portion 214 of the tower access device 200 and the slot 612 engages the rod 118. As the top portion 620 moves distally on the proximal threaded portion 214, the slot 612 engages the rod 118 so that the cannula 610 urges the rod 118 into position with respect to the housing 112.

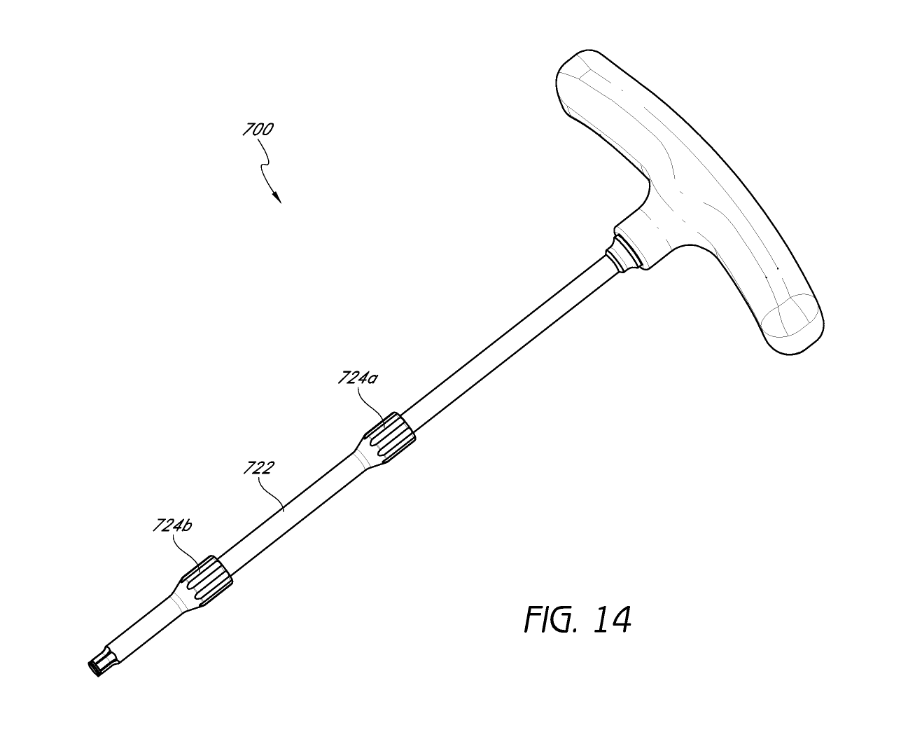

As described herein, a spinal stabilization system 100 can include a set screw 116 to fix the rod 118 and spinal screw 110. A set screw inserter 700 can be provided and used to thread the set screw 116 into the internal threads 126 of the housing 112. As shown in FIG. 14, the set screw inserter 700 can have two load bearing portions 724a,b and a reduced diameter shaft 722 similar to the dilation tubes 300, awl 320, and tap 330 described herein to advantageously reduce the weight of the inserter 700.

### Hinge For Tower Access Devices

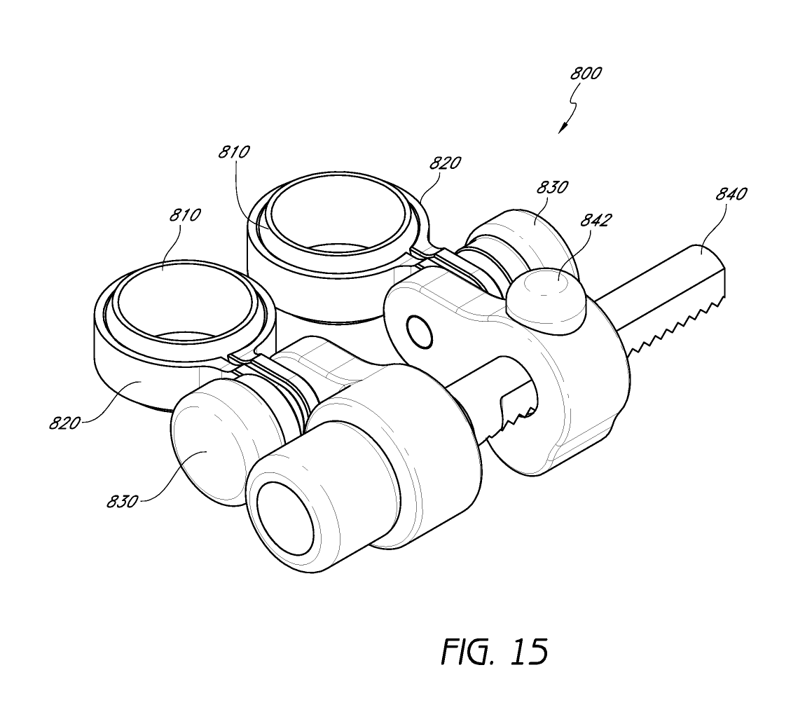

In some spinal stabilization procedures, two or more spinal stabilization systems 100 are implanted into adjacent vertebrae and connected by one or more rods 118. Two or more tower access devices 200 can be used to insert the systems 100. A hinge 800 as shown in FIG. 15 can be used to couple two tower access devices 200, for example, to hold them in place relative to each or to allow for compression or distraction of the vertebrae. In the illustrated embodiment, the hinge 800 includes two pivots 810, two clamshell clamps 820, two clamp tighteners 830, a ratchet bar 840, and a tightening knob 842. The pivots 810 have spherical outer surfaces and cylindrical bores therethrough. The pivots 810 are sized and configured to slide onto the tower access devices 200. The clamshell clamps 820 circumferentially surround and are configured to be tightened around the pivots 810. The clamshell clamps 820 have spherical inner surfaces configured to engage the spherical outer surfaces of the pivots 810. The spherical surfaces allow the pivots 810 to pivot and angulate relative to the clamshell clamps 820 with multiple degrees of freedom. The clamp tighteners 830 can be tightened to secure the clamshell clamps 820 around the pivots 810 and adjust the amount of movement allowed by the pivots 810. The ratchet bar 840 can be used to adjust the lateral spacing between the clamshell clamps 820 to match the spacing of the towers 200. When the desired spacing is achieved, the ratchet bar 840 can be locked via the tightening knob 842 to help hold the towers 200 in place. The hinge 800 can also advantageously serve as a fulcrum for compression or distraction. In some embodiments, a hinge having more than two pivots 810 and clamshell clamps 820 can be used to couple more than two access devices 200.

### Other Instruments

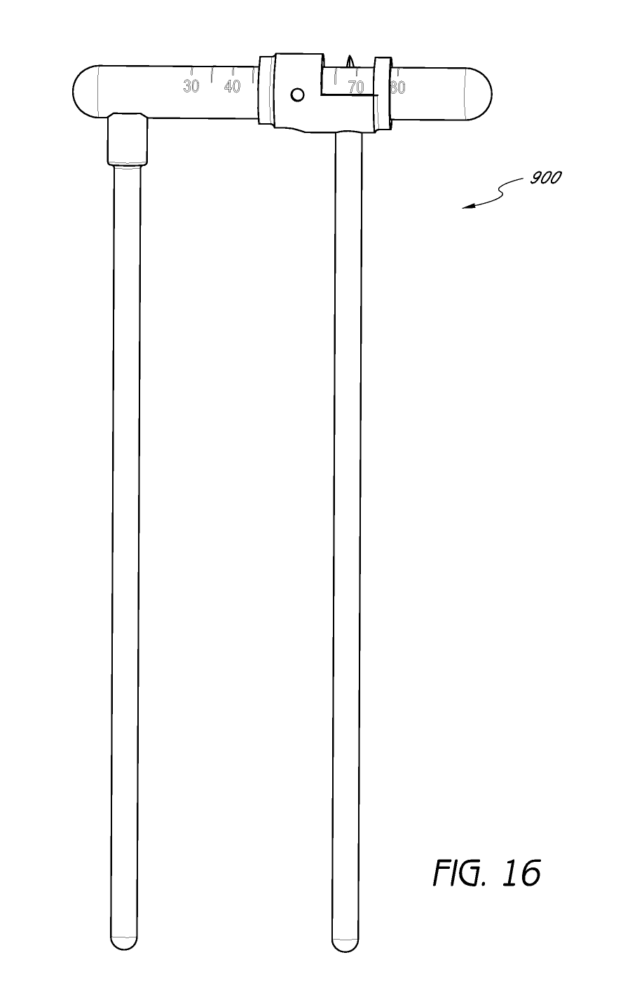

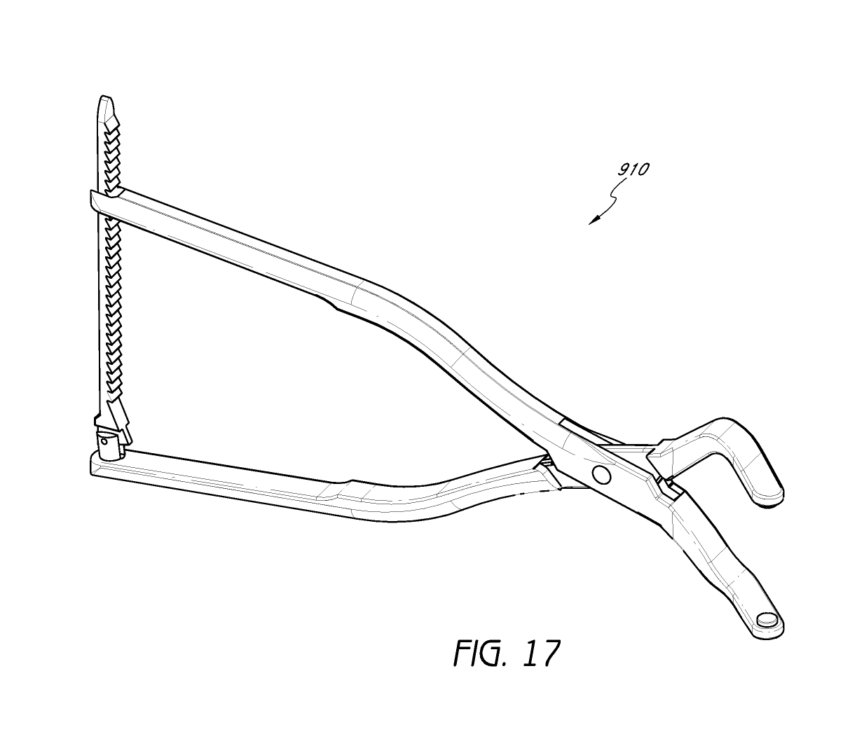

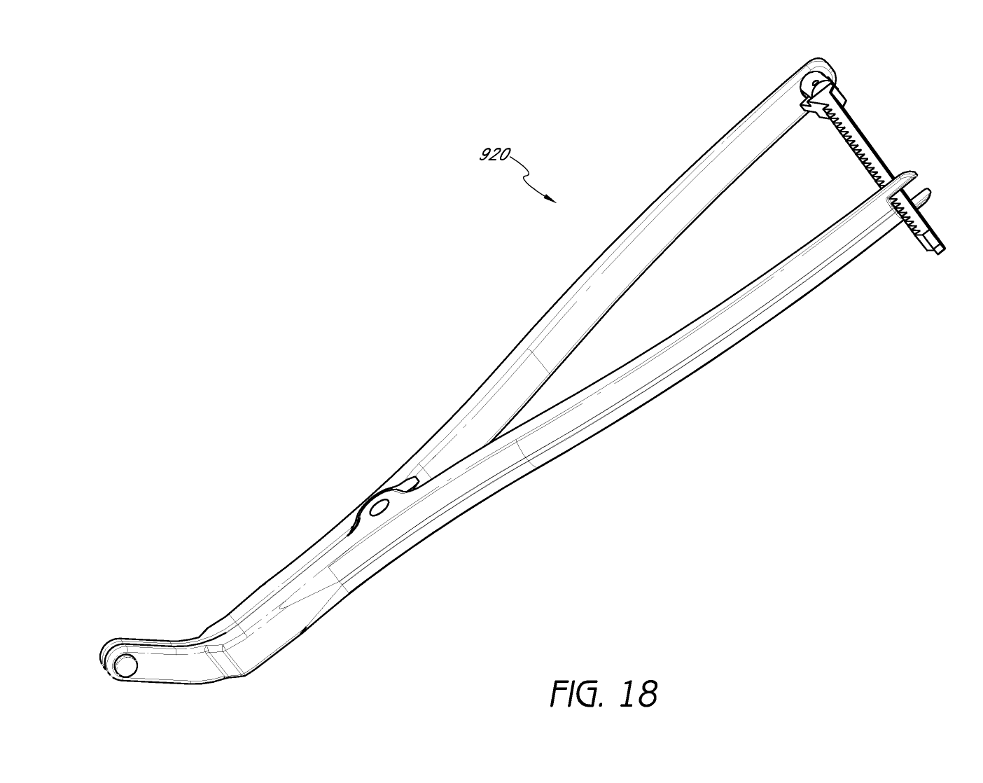

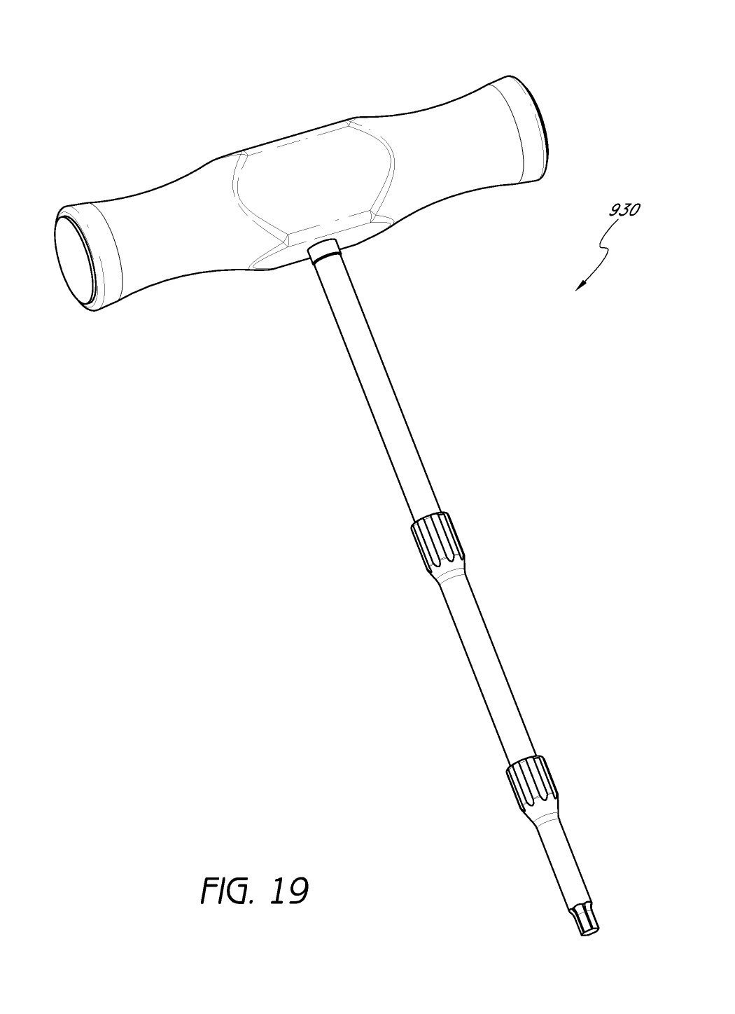

Various other instruments can also be provided. For example, a caliper 900, as shown in FIG. 16, can be used to measure the length needed for the rod 118. A compressor 910 and/or distractor 920, as shown in FIGS. 17 and 18, respectively, allow for adjustment of the lateral spacing of the towers and therefore allow for compression or distraction of the vertebrae. A final tightener 930, shown in FIG. 19 and similar to set screw inserter 700, can be provided for final tightening of the set screw 116 once the desired spacing of the spinal stabilization systems 100 has been achieved. The final tightener 930 can have a T-handle as shown in FIG. 19 or another type of handle, e.g., a palm, trilobe, axial, or other handle.

### Systems or Kits

In some embodiments, one or more of the instruments described herein can be packaged together in a kit or system, for example by packaging one or more instruments in a single tray. For example, a kit or system may include one or more of: dilation tubes 300, an awl 320, a tap 330, a screw delivery device 400, a rod inserter 500, a threaded reducer 600, and tower access devices 200. A kit or system can further include a set screw inserter 700 and/or final tightener 930. In some embodiments, a kit or system can include a compressor 910 and/or distracter 920. Furthermore, in some embodiments, a kit or system can include a Jamshidi needle and/or a K-wire. In some embodiments, the kit or system may further include implants such as screws and rods and may include a caliper 900.

Method for Delivering Fixation Device or Implant into a Spine

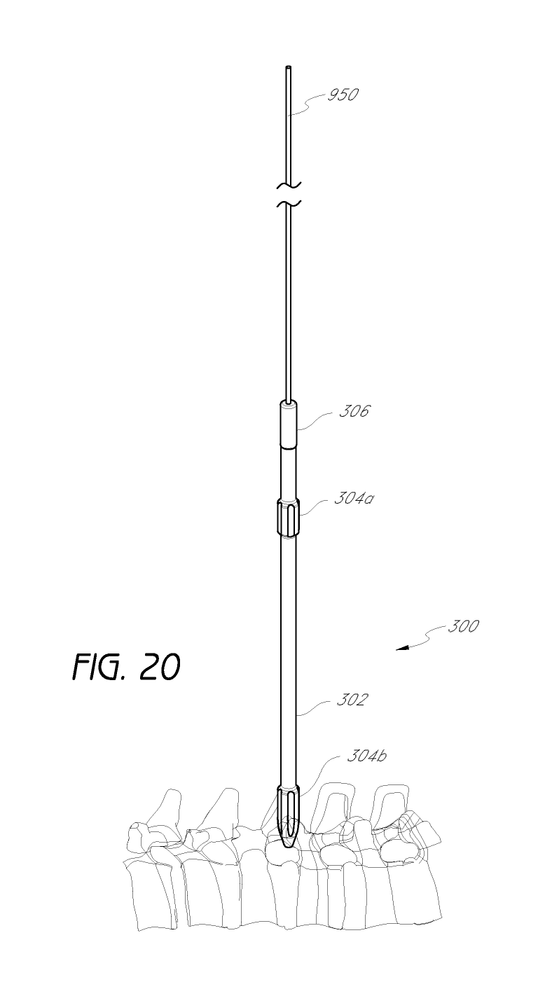

FIGS. 20-33 show an example embodiment of a method for delivering a fixation device or implant into a spine for spinal stabilization using various instruments as described herein. To begin, the surgeon makes a small incision through the patient's skin at the desired insertion site and inserts a Jamshidi needle through the skin to the bone surface. Once the Jamshidi needle is docked at the desired insertion location, for example, the junction of the transverse process and facet joint for a pedicle screw insertion procedure, the inner stylet is removed and a K-wire is inserted into the vertebral body. The K-wire is then used to guide a series of dilation tubes of increasing diameter that sequentially enlarge the working channel. FIG. 20 illustrates a dilation tube 300 being delivered over a K-wire 950.

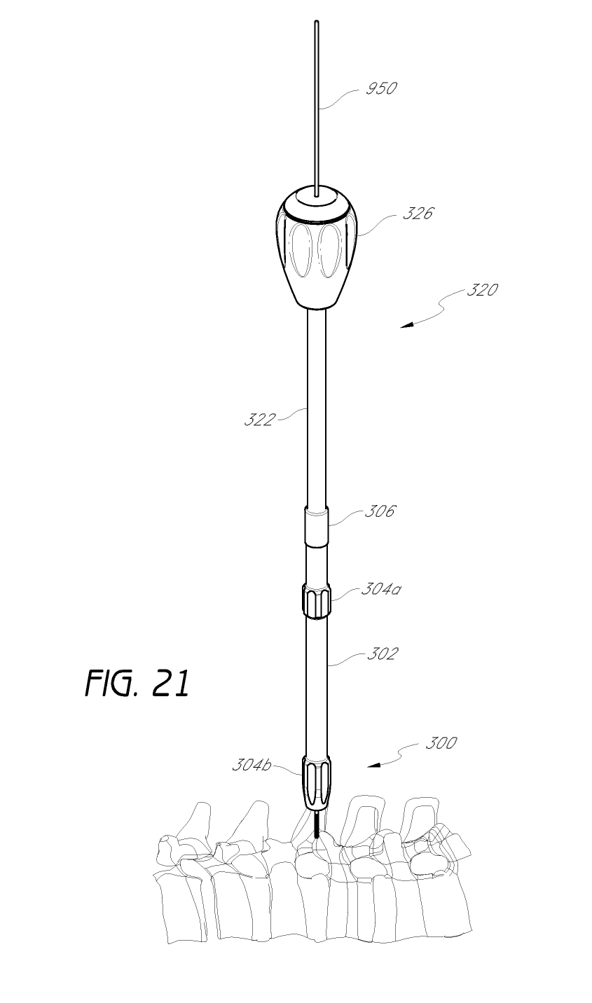

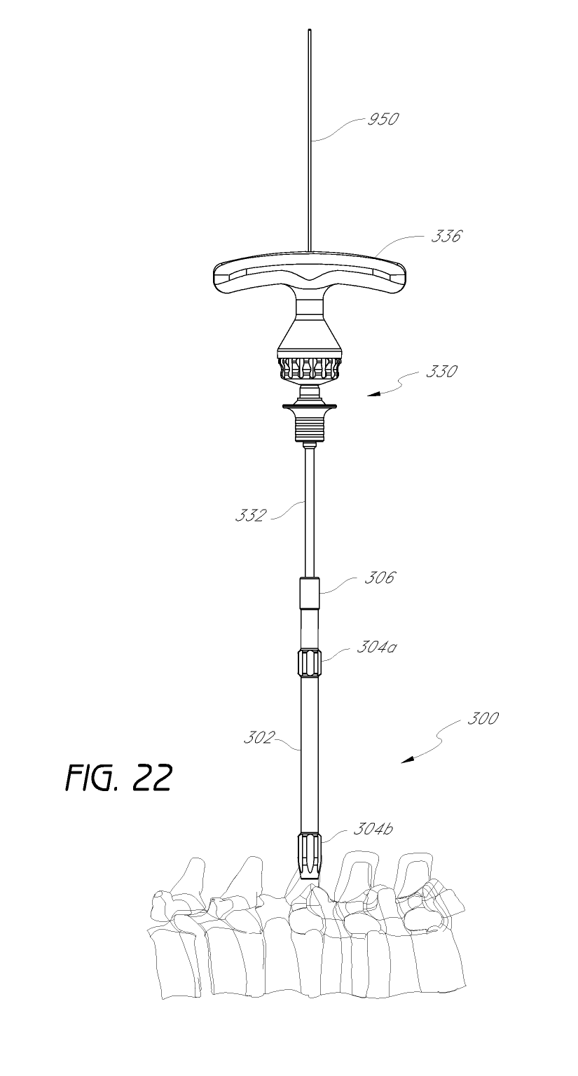

The working channel to the patient's spine can be dilated to the desired size by using progressively larger dilation tubes. In some embodiments, the surgeon can use traditional dilator tubes to gradually enlarge the working channel and then insert a dilation tube 300 having the improved shape described herein as the final dilation tube 300 through which other instruments can be inserted. Alternatively, multiple dilation tubes 300 of different sizes can be used. The surgeon can use dilation tubes 300 to introduce instruments such as an awl 320, drill, and/or tap 330 to the insertion site to prepare the bone for the implant, for example as shown in FIGS. 21 and 22. Different sized dilation tubes 300 can be used to introduce different instruments.

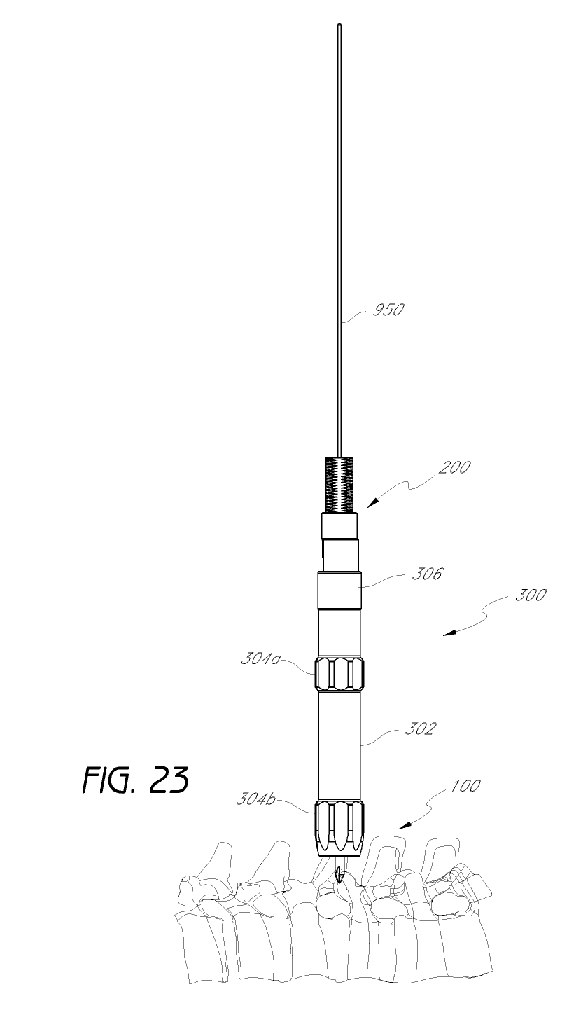

Once the insertion site has been prepared, the surgeon can couple an implant, for example, the screw and housing of a spinal stabilization system 100, to the tower access device 200 by engaging the protruding members 216 of the grasping elements 212 with the apertures 124 on the housing 112. The lock nut 240 is rotated clockwise on the threaded portion 214 of the inner sleeve 210 to move the outer sleeve 220 relative to the inner sleeve 210, compress the grasping elements 212 about the housing 112, and lock the assembly. The surgeon can then introduce the assembly to the insertion site through the dilation tube 300 as shown in FIG. 23. Alternatively, the dilation tube 300 can be removed prior to introducing the tower access device 200, and the tower access device 200 can be tracked over the K-wire.

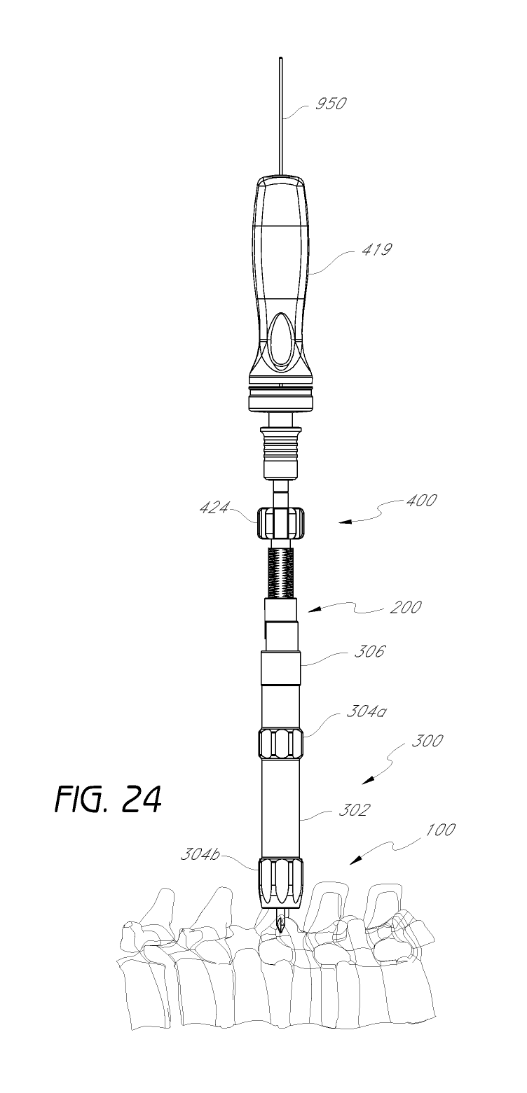

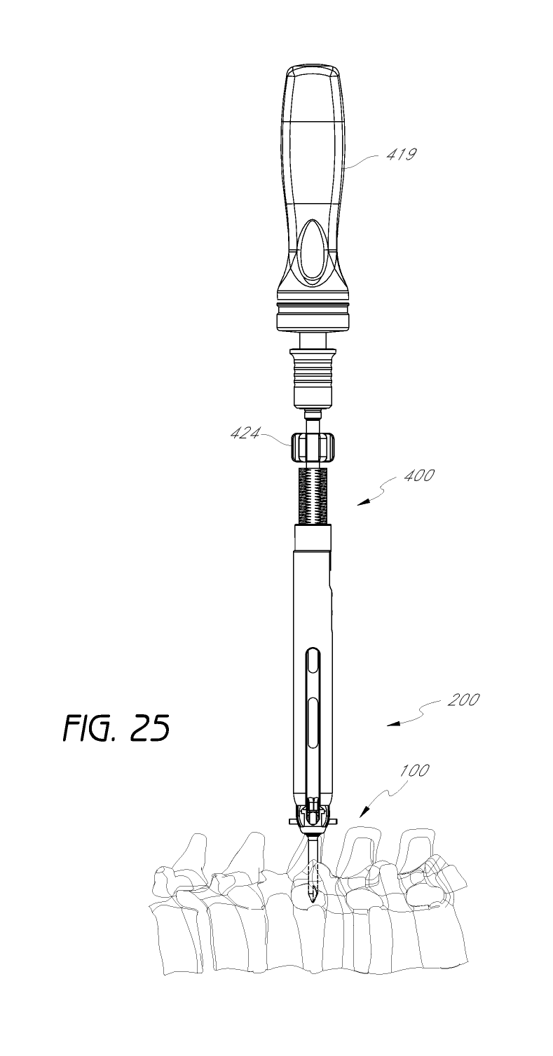

The screw 110 can be inserted into the patient's vertebra using the screw delivery device 400 introduced through the tower access device 200, which in turn can be introduced through the dilation tube 300 as shown in FIG. 24. In FIG. 24, the screw delivery device 400 is illustrated including a trilobe handle 419. Other types of handles are also possible as described herein. Alternatively, the dilation tube 300 can be removed prior to introducing the screw delivery device 400, for example as shown in FIG. 25.

One or more screws can be fixed into bone using the devices and methods described above. For example, in one embodiment, a first screw attached to a first access device can be delivered into a first vertebrae, while a second screw attached to a second access device can be delivered into a second vertebrae.

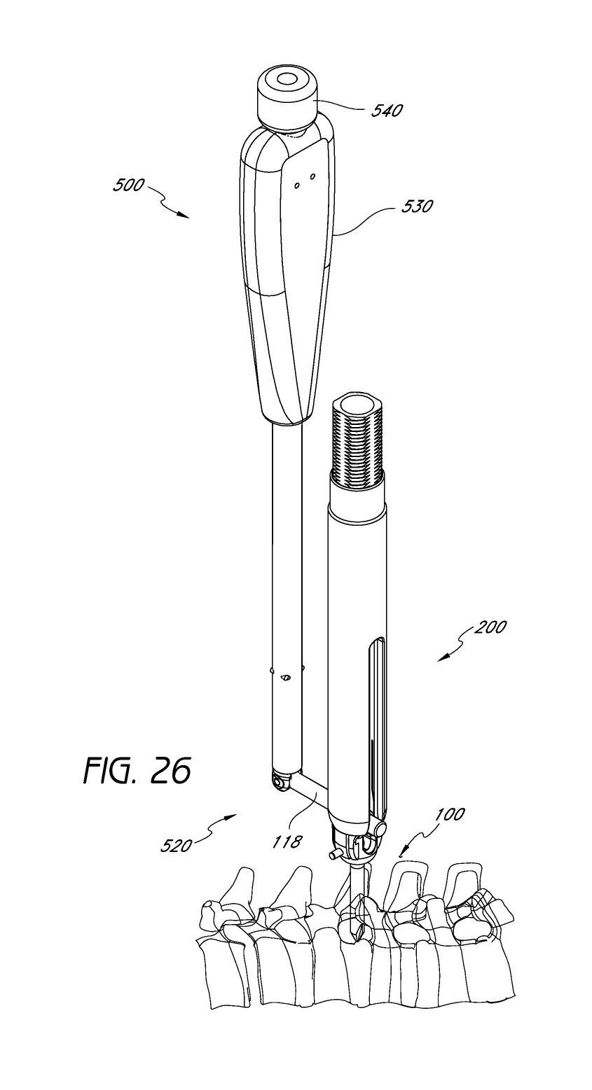

Once the desired number of screws and access devices are delivered, the screw delivery device 400 and dilation tube 300 can be removed and the rod 118 can be inserted using the rod insertion device 500 as shown in FIG. 26. As described herein, the actuator knob 540 can be pulled proximally to cause the gripping end 520 of the rod insertion device 500 to grip the rod 118. The pins and serrations 522 can enhance the grip. In some embodiments, the rod 118 is delivered via a mini-open procedure, in which an incision is made between the first and second screw. In other embodiments, the rod 118 is delivered percutaneously along the side of either of the first and second access devices. The rod 118 can be delivered along the outer sidewall of a first access device. The rod 118 can be delivered at an angle such that a first end is received into a distal slot 211, 221 of the first access device 200. The rod 118 can then be directed such that its second end is received into a distal slot 211, 221 of the second access device 200. One end of the rod 118 can be fixed to the first screw, while the opposite end of the rod member can be fixed to the second screw, thereby forming a stabilizing connection between the screws. The surgeon can advantageously release the actuator knob 540 halfway to loosen the grip of the gripping end 520 on the rod 118 while leaving the pins in place to assist in adjusting the rod 118 to the desired position. This allows the surgeon to manipulate and change an angle of the rod insertion device 500 relative to the rod 118 and/or the rod 118 relative to the access device 200 as needed to deliver the rod 118 to the desired position.

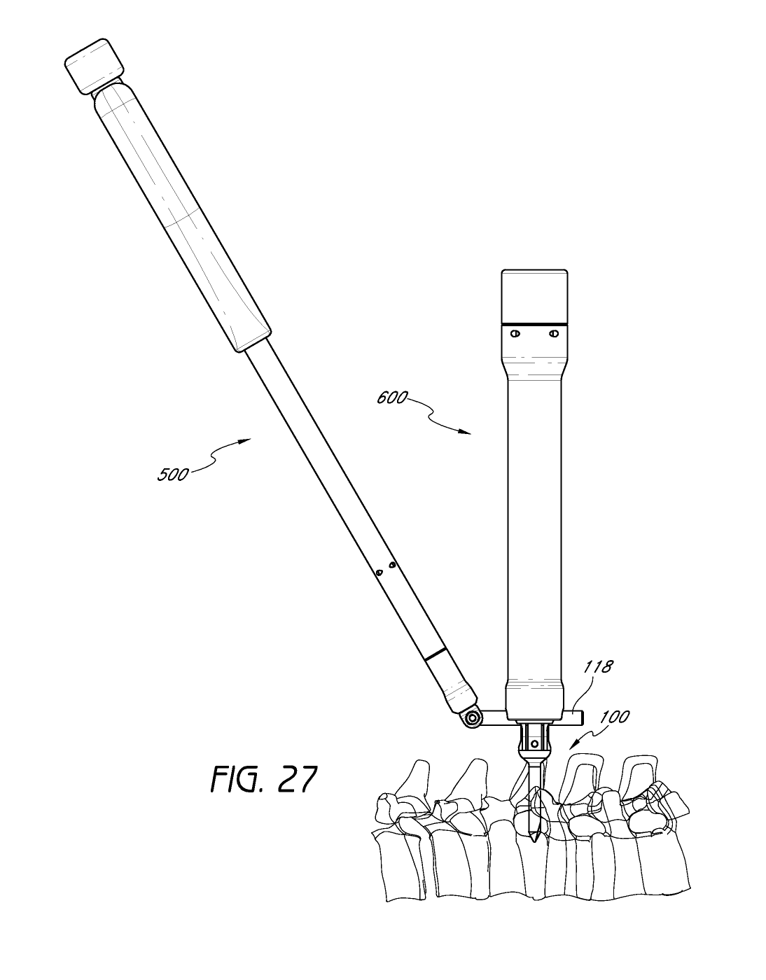

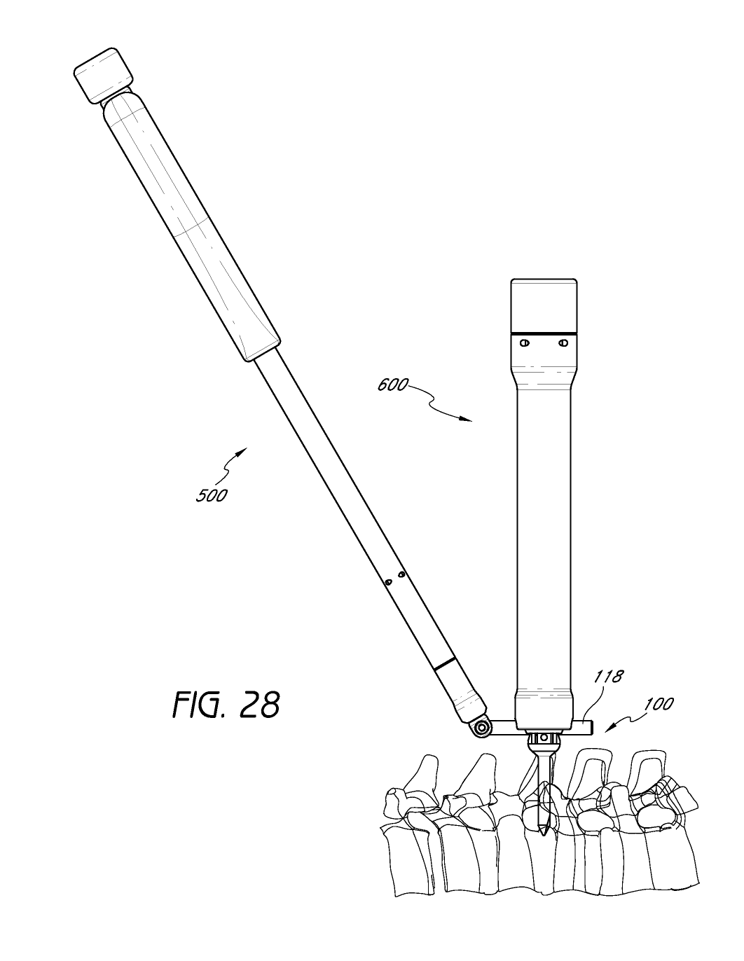

The threaded reducer 600 can then be introduced over the tower access device 200 so that the slot 612 engages the rod 118 as shown in FIG. 27. As the threaded reducer 600 is threaded onto the inner sleeve 210 of the tower access device 200, the rod 118 is forced into contact with the one or more clamps 114 of the system 100 as shown in FIG. 28. The rod insertion device 500 may be left in place during reduction with the threaded reducer 600 to help the surgeon maintain the rod 118 in the desired location.

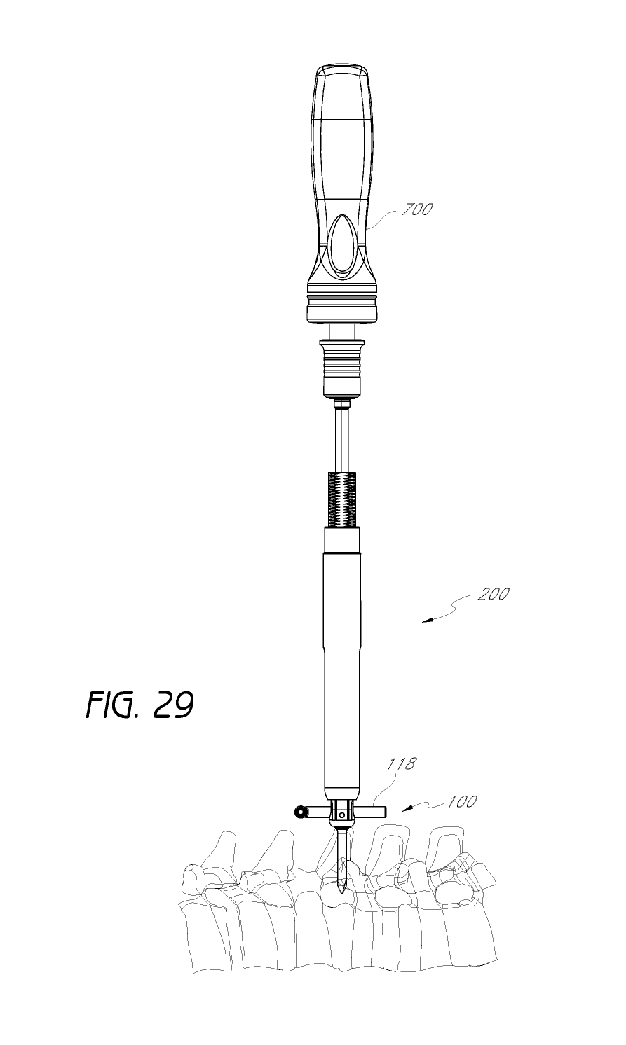

Once the rod 118 is in place, the rod insertion device 500 can be removed, and a set screw inserter 700 can be inserted through the tower access device 200 to thread the set screw 116 into the housing 112 to secure the screw 110 rod 118 as shown in FIG. 29. The set screw 116 can provide a downward force on the rod 118, and this downward force can be transferred from the rod 118 to the clamps 114 and housing 112, thereby providing a secure locking mechanism for the system.

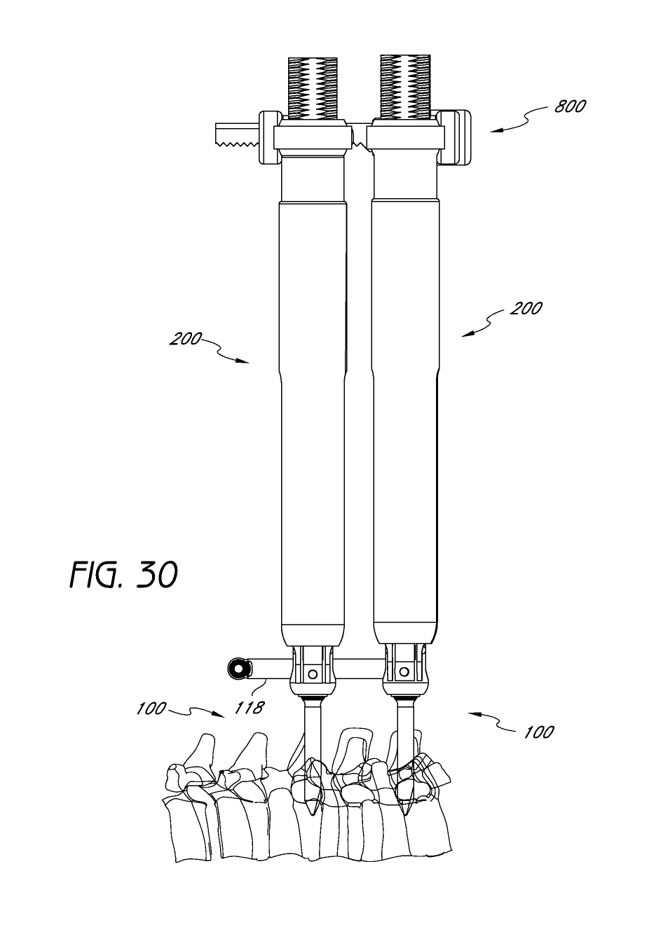

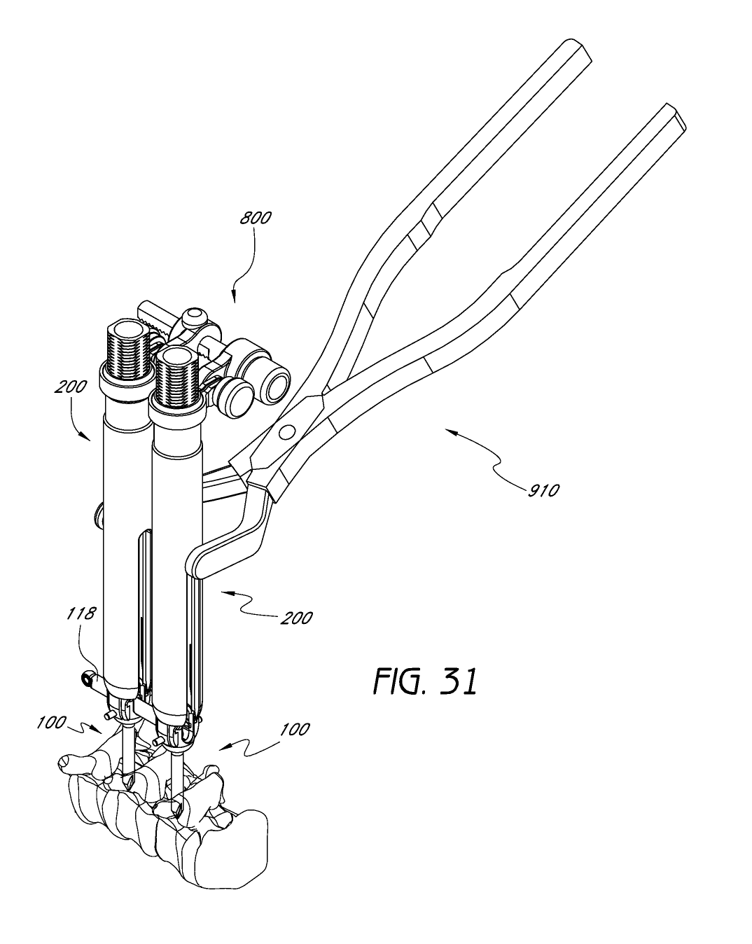

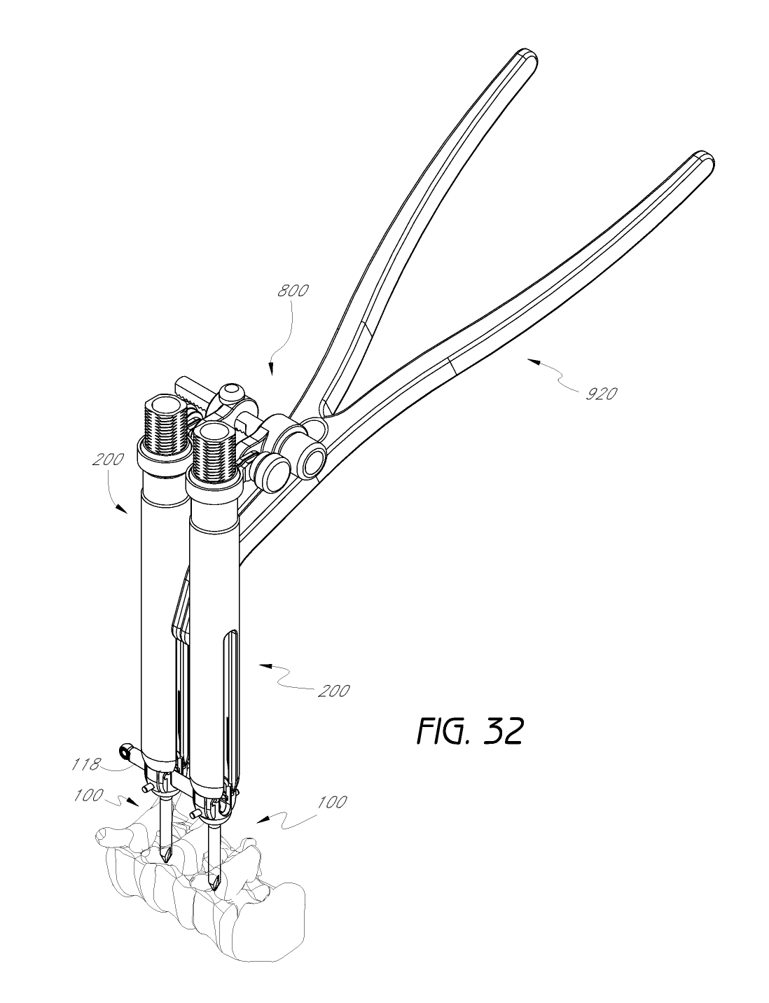

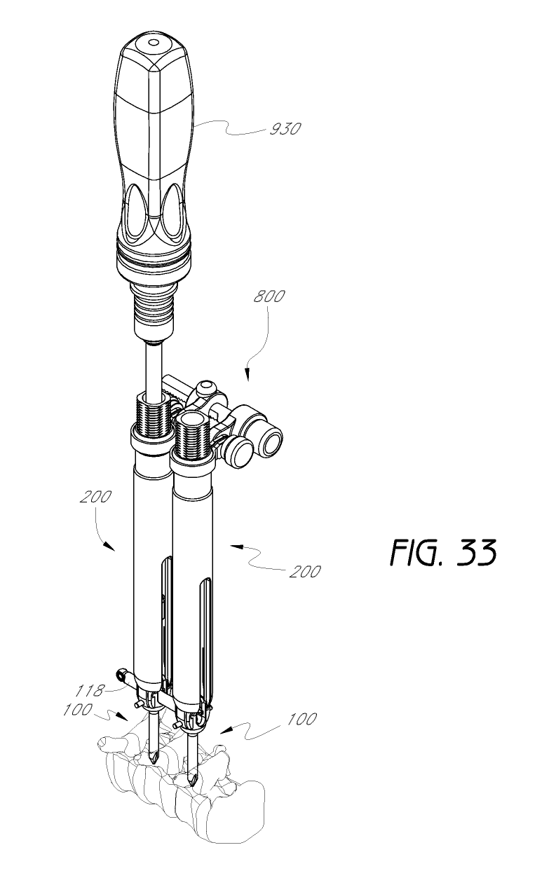

As described herein, multiple tower access devices 200 can be used to insert multiple systems 100 into the patient's spine. For example, additional tower access devices 200 and spinal stabilization systems 100 can be placed in adjacent locations such as the pedicles of adjacent vertebra. The rod 118 then spans the systems 100 to stabilize the spine as shown in FIG. 30. The hinge 800 can be used to secure the towers 200 relative to each other while allowing some pivotal movement. If desired, a compressor 910 or distracter 920 can be used to force adjacent tower access devices 200 closer to or farther apart from one another and in turn compress or distract the screws 110 and the patient's spine as shown in FIGS. 31 and 32, respectively. The hinge 800 can advantageously act as a fulcrum for such distraction or compression. Finally, the surgeon can perform final tightening with a final tightener 930 or screwdriver as needed, as shown in FIG. 33.

Once the implants are in place, the surgeon can release and remove the hinge 800. The surgeon can then release the grasping elements 212 of the access devices 200 from the housings 112 by depressing the spring latch 230 button 232 to release the engagement of the spring latch 230 tab 234 and lock nut 240 engagement surface 244. The lock nut 240 can then be threaded off of the access device 200 so the access device(s) 200 can be removed.

It will be apparent to those skilled in the art that various modifications and variations can be made in the present embodiments without departing from the scope or spirit of the advantages of the present application. Thus, it is intended that the present application cover the modifications and variations of these embodiments and their equivalents.

1. (canceled)

2. (canceled)

3. (canceled)

4. (canceled)

5. (canceled)

6. (canceled)

7. (canceled)

8. (canceled)

9. A method of delivering a screw assembly to a spinal location, comprising:
positioning a screw assembly at a desired spinal location, the screw assembly comprising:
a housing having a lower opening and internal threads configured to receive an externally threaded set screw;
a threaded shaft having an enlarged head, the threaded shaft extending through the lower opening and the enlarged head sitting adjacent the lower opening; and
a rod receiving surface within the housing having a concave shape configured to receive a rod;
wherein the threaded shaft is rotatable relative to the housing prior to insertion of the set screw;
engaging a screw delivery device with the screw assembly at the spinal location, the screw delivery device comprising:
an inner elongate shaft having a proximal end and a distal end, the distal end of the inner shaft comprising a hexalobe tip configured to engage a corresponding opening in the enlarged head of the screw assembly and a saddle proximal to the hexalobe tip configured to engage the rod receiving surface;
an outer elongate shaft provided over the inner elongate shaft, the outer shaft being axially and rotationally moveable relative the inner shaft, the outer shaft having a proximal end and a distal end, the proximal end of the outer shaft comprising a grip and the distal end of the outer shaft comprising an externally threaded portion configured to engaged the internal threads of the housing;
rotating the outer shaft relative to the inner shaft while the hexalobe tip engages the enlarged head of the screw assembly to cause the externally threaded portion at the distal end of the outer shaft to engage the internal threads of the housing; and
rotating the screw delivery device while the inner and outer shafts remain relatively locked in position to drive the threaded shaft into the spinal location, said rotation rotating the hexalobe tip against the opening in the enlarged head of the screw assembly with the saddle distributing load to the rod receiving surface during said rotation.

10. A rod insertion device, comprising:
an elongate, generally cylindrical shaft;
a gripping end at a distal end of the shaft, wherein the gripping end includes two or more gripping elements configured to grip a rod member;
a handle at a proximal end of the shaft; and
an actuator member configured to cause relative movement between the shaft and gripping end, wherein distal movement of the shaft or proximal movement of the gripping end causes compression of the gripping elements.

11. The rod insertion device of claim 10, wherein the gripping end comprises serrations configured to mate with serrations on the rod member.

12. A system comprising the rod insertion device of claim 11 and a rod member having serrations configured to mate with the serrations on the gripping end.

13. A rod insertion device, comprising:
an elongate shaft having a proximal end and a distal end;
a gripping end at a distal end of the shaft, wherein the gripping end includes two or more gripping elements configured to grip a rod member; and
an actuator configured to compress the gripping elements onto an elongate rod member;
wherein the gripping elements comprise serrations configured to mate with serrations on the rod member.

14. The rod insertion device of claim 13, wherein the gripping elements comprise at least one pin configured to engage a hole in the rod member.

15. The rod insertion device of claim 13, comprising an outer shaft and an inner shaft, the gripping end provided at a distal end of the inner shaft, and wherein the outer shaft is moveable over the inner shaft to compress the gripping elements.

16. A system comprising the rod insertion device of claim 13 and a rod member having serrations configured to mate with the serrations on the gripping elements.

17. A system comprising the rod insertion device of claim 14 and a rod member having serrations configured to mate with the serrations on the gripping elements and a hole configured to receive the at least one pin.

18. A rod for use for spinal fixation, the rod comprising an elongate cylindrical member having a first end and a second end, wherein one of the ends comprises a plurality of serrations.

19. The rod of claim 18, further comprising a hole extending through the end having the plurality of serrations.

20. A method of delivering a rod to a spinal location, comprising:
providing a rod having a serrated portion;
providing a rod insertion device comprising:
an elongate, generally cylindrical shaft having a gripping end at a distal end of the shaft and a handle at a proximal end of the shaft;
wherein the gripping end includes two or more gripping elements configured to grip the rod member, the gripping elements having serrations;
grasping a rod with the rod insertion device by engaging the serrations of the gripping elements and serrated portion of the rod; and
delivering the rod to the spinal location with the rod insertion device.

21. The method of claim 20, wherein the rod has at least one aperture proximate the serrated portion, and the rod insertion device comprises one or more pins that engage the at least one aperture.

22. The method of 21, further comprising adjusting a position of the rod by loosening the engagement of the serrations of the gripping elements and serrated portion of the rod while leaving the one or more pins in the at least one aperture.

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)
